(19) United States
(12) Patent Application Publication
(10) Pub. No.: US 2003/0017162 A1
(43) Pub. Date: Jan. 23, 2003

(54) Outer membrane protein A, peptidoglycan-associated lipoprotein, and murein lipoprotein as therapeutic targets for treatment of sepsis

(76) Inventors: H. Shaw Warren, Charlestown, MA (US); Judith Hellman, Brookline, MA (US); James T. Kurnick, Winchester, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/097,538

(22) Filed: Mar. 13, 2002

Related U.S. Application Data
(63) Continuation of application No. 09/641,620, filed on Aug. 18, 2000, now abandoned.
(60) Provisional application No. 60/149,960, filed on Aug. 20, 1999.

(51) Int. Cl. A61K 39/40; A61K 39/02

(52) U.S. Cl. 424/164.1; 424/190.1

(57) ABSTRACT

The present invention relates to three outer membrane proteins conserved among Gram-negative bacteria, OmpA, PAL, and MLP. The invention provides vaccines and polypeptides useful for passive and active immunization against Gram-negative bacteria, as well as methods of preventing and treating Gram-negative sepsis.

### RELATED APPLICATION

This application is a Continuation of Ser. No. 09/641,620, filed Aug. 18, 2000, which claims the benefit of U.S. Provisional Patent Application No. 60/149,960, filed Aug. 20, 1999. The entire teachings of the above applications are incorporated herein by reference.

### GOVERNMENT RIGHTS

This invention was funded in part under National Institute of Health Grant No. ROA13967-01. The government may retain certain rights in these inventions.

### FIELD OF INVENTION

The present invention relates to pharmaceutical compositions and methods useful for preventing and treating Gram-negative sepsis. In particular, the invention arises from the identification of three outer membrane proteins conserved among a number of Gram-negative bacteria and relates to antibodies directed to them.

### BACKGROUND OF INVENTION

Infections due to Gram-negative bacteria are a major cause of morbidity and mortality. Gram-negative sepsis, the systemic inflammatory response to the microbial invasion, often first manifested as fever, hypothermia, tachycardia, or tachypnea, can progress to life-threatening hypotension and organ failure. While microbial invasion of the bloodstream is common in advanced stages of sepsis, localized Gram-negative infections can lead to Gram-negative sepsis on the basis of host responses to local or systemic release of microbial signals. Such microbial signals frequently arise from bacterial cell wall components such as lipopolysaccharide (LPS), also known as endotoxin.

The notion of treating Gram-negative sepsis with antibody directed to conserved cell wall components is supported by many studies over the last thirty years that show that administration of polyclonal antisera raised to rough mutant bacteria protects in Gram-negative sepsis caused by heterologous Gram-negative bacteria. Chedid L et al., A proposed mechanism for natural immunity to enterobacterial pathogens, J Immunol 100:292-301 (1968); Braude Al et al., Treatment and prevention of intravascular coagulation with antiserum to endotoxin, J Infect Dis 128:S 157-S 164 (1973); McCabe W R et al., Cross-reactive antigens: Their potential for immunization-induced immunity to gram-negative bacteria, J Infect Dis 136:S161-S 166 (1977); McCabe W R et al., Immunization with rough mutants of Salmonella minnesota: protective activity of IgM and IgG antibody to the R595 (Re Chemotype) mutant, J Infect Dis 158:291-300 (1988); Ziegler E J et al., Treatment of gram-negative bacteremia and shock with human antiserum to a mutant Escherichia coli, N Engl J Med 307:1225-1230 (1982); Baumgartner J et al., Prevention of Gram-negative shock and death in surgical patients by antibody to endotoxin core glycolipid, Lancet 59-63 (1985).

It has generally been assumed that immunoglobulins in antisera to rough mutant strains such as Escherichia coli J5 and Salmonella minnesota Re595 protect by binding to conserved core components (lipid A and core oligosaccharide) of lipopolysaccharide (LPS). There has not, however, been direct evidence that anti-core monoclonal antibodies protect, with the exception of one monoclonal antibody, WN1 222-5, which has been reported to bind core structures of LPS from heterologous enteric Gram-negative bacteria and to protect from endotoxin challenge in rabbits and mice. Di Padova FE et al., A broadly cross-protective monoclonal antibody binding to Escherichia coli and Salmonella lipopolysaccharides, Infect Immun 61:3863-3872 (1993). In addition, it has been difficult to directly demonstrate substantial increased binding to LPS from heterologous Gram-negatives by the immunoglobulins in polyclonal antiserum to E. coli J5. Siber G R et al., Cross-reactivity of rabbit antibodies to lipopolysaccharides of Escherichia coli J5 and other gram-negative bacteria, J Infect Dis 152:954-964 (1985); Warren HS et al., Endotoxin neutralization with rabbit antisera to Escherichia coli J5 and other gram-negative bacteria, Infect Immun 55:1668-1673 (1987). Nonetheless, although antisera raised to heat-killed rough strains have been reported to protect, the exact mechanism by which this protection occurs remains elusive.

We previously reported that immunoglobulin G (IgG) in these antisera bind only weakly to LPS from heterologous Gram-negative strains. Siber G R et al., Cross-reactivity of rabbit antibodies to lipopolysaccharides of Escherichia coli J5 and other gram-negative bacteria, J Infect Dis 152:954-964 (1985); Warren HS et al., Endotoxin neutralization with rabbit antisera to Escherichia coli J5 and other gram-negative bacteria, Infect Immun 55:1668-1673 (1987). The resounding clinical failure of anti-lipid A monoclonal antibodies (that were based upon these antisera) has resulted in decreased interest in this approach.

We also recently reported that IgG in polyclonal antiserum raised to heat-killed E. coli J5 bacteria (J5 antiserum) binds to three conserved Gram-negative bacterial outer membrane proteins (OMPs). These OMPs are exposed on the surface of bacteria incubated in human serum and are released into human serum in complexes that also contain LPS. Hellman J et al., Antiserum against Escherichia coli J5 contains antibodies reactive with outer membrane proteins of heterologous Gram-negative bacteria, J Infect Dis 176:1260-1268 (1997). The identities of the antigens bound by J5 antiserum are unknown.

### SUMMARY OF THE INVENTION

The invention solves these and other problems by providing methods and compositions for treating infection and sepsis due to Gram-negative bacteria.

In a first aspect the invention provides a vaccine composition comprising an effective amount of an isolated outer membrane protein (OMP) selected from the group consisting of outer membrane protein A (OmpA), peptidoglycan-associated lipoprotein (PAL), murein lipoprotein (MLP), and immunogenic portions thereof, in a pharmaceutically suitable carrier. The vaccine is believed to be useful for active immunization against multiple Gram-negative bacteria. The vaccine can include an adjuvant, which can preferably be selected from Al(OH)₃, AlPO₄, QS21, CpG, and any combination of these.

In one embodiment the isolated OMP is OmpA. In another embodiment the isolated OMP is PAL. In yet another embodiment the isolated OMP is MLP.

In another aspect the invention provides an adjuvant comprising an effective amount of an isolated OMP selected from the group consisting of OmpA, PAL, MLP, and any combination thereof, in a pharmaceutically acceptable carrier. The adjuvant can be used in association with exposure to an antigen other than OmpA, PAL, MLP, and immunogenic portions thereof.

The invention in another aspect provides a pharmaceutical composition comprising an effective amount of an isolated polypeptide that binds specifically to at least a portion of OmpA, PAL, or MLP, in a pharmaceutically suitable carrier. In various embodiments the isolated polypeptide can include a monoclonal antibody, a derivative of a monoclonal antibody, a polyclonal antibody, or a synthetic polypeptide. The antibody can be a human antibody or a humanized antibody. Preferably the antibody or antibody derivative is a human antibody. The polyclonal antibody is distinct from polyclonal antibody raised against killed whole Gram-negative bacteria and unfractionated cell walls from Gram-negative bacteria. Preferably the synthetic polypeptide is a member of a combinatorial library of synthetic polypeptides.

In yet another aspect the invention provides an immortal cell line that secretes a polypeptide that binds specifically to an outer membrane protein selected from the group consisting of OmpA, PAL, MLP, and any immunogenic portion thereof. In certain embodiments the secreted polypeptide is a monoclonal antibody. In other embodiments the secreted polypeptide includes a fragment of a monoclonal antibody. In preferred embodiments the monoclonal antibody or fragment of a monoclonal antibody is of human origin. In alternative preferred embodiments the monoclonal antibody or fragment of a monoclonal antibody is humanized.

In one embodiment of this aspect of the invention the isolated OMP is OmpA. In another embodiment the isolated OMP is PAL. In yet another embodiment the isolated OMP is MLP.

Another aspect of the invention is a method of immunizing a subject against infection due to Gram-negative bacteria wherein a subject is administered an isolated outer membrane protein antigen selected from the group consisting of OmpA, PAL, MLP, and any immunogenic portion thereof, in a pharmaceutically suitable carrier, in an amount effective for inducing protection against infection due to Gram-negative bacteria. In one embodiment of this aspect of the invention the isolated OMP is OmpA. In another embodiment the isolated OMP is PAL. In yet another embodiment the isolated OMP is MLP. In a further embodiment the methods of active vaccination can include administration of an adjuvant. Preferably the adjuvant is selected from Al(OH)₃, AlPO₄, QS21, CpG, and any combination of these. In certain embodiments the antigen is administered subcutaneously. In alternative embodiments, the antigen is administered intradermally, intramuscularly, or mucosally.

In another aspect the invention provides a method of treating a subject infected with Gram-negative bacteria, wherein the method involves administering to a subject who has an infection with Gram-negative bacteria an isolated polypeptide that binds specifically to at least a portion of an outer membrane protein selected from the group consisting of OmpA, PAL, and MLP, in an amount effective to treat the infection. In a preferred embodiment the amount is effective to inhibit Gram-negative sepsis. In another preferred embodiment the amount is effective to inhibit growth of the Gram-negative bacteria in vivo.

In various embodiments of this aspect of the invention, the isolated polypeptide can be a monoclonal antibody, a derivative of a monoclonal antibody, a polyclonal antibody, or a member of a library of synthetic polypeptides.

In certain embodiments the administered amount of polypeptide is effective to enhance clearance of Gram-negative bacteria from blood of the subject. In other embodiments the administered amount of polypeptide is effective to enhance clearance of insoluble fragments of Gram-negative bacteria from blood of the subject.

In yet other embodiments the administered amount of polypeptide is effective to neutralize Gram-negative bacteria in blood of the subject. In other embodiments the administered amount of polypeptide is effective to neutralize insoluble fragments of Gram-negative bacteria in blood of the subject.

According to another embodiment the administered amount of polypeptide is effective to opsonize Gram-negative bacteria in blood of the subject. In a further embodiment the administered amount of polypeptide is effective to opsonize insoluble fragments of Gram-negative bacteria in blood of the subject.

In certain embodiments the method also involves administration of an effective amount of an immune system stimulant. In preferred embodiments the immune system stimulant is a cytokine. In other preferred embodiments the immune system stimulant is an adjuvant.

In a further aspect the invention provides a method of treating a subject with Gram-negative sepsis, wherein a subject in need of such treatment is administered a composition containing an isolated polypeptide that binds specifically to at least a portion of an outer membrane protein selected from the group consisting of OmpA, PAL, and MLP, in an amount effective to inhibit sepsis-related release of at least one soluble factor into blood or tissue of the subject.

In certain embodiments of this aspect of the invention, the soluble factor is released by Gram-negative bacteria upon their exposure to serum. In one embodiment the soluble factor is LPS. In another embodiment the soluble factor is OmpA. In a further embodiment the soluble factor is PAL. In yet another embodiment the soluble factor is MLP.

In certain other embodiments of this aspect of the invention, the soluble factor is released by cells of the infected host. In some embodiments the soluble factor is a cytokine. In yet other embodiments the released factor is selected from IL-1, IL-6, TNF-α, high mobility group-1 protein (HMG-1), migration inhibitory factor (MIF), chemokines, and nitric oxide.

In yet a further aspect the invention provides a method of treating a subject who has Gram-negative sepsis, involving administering to a subject in need of such treatment a composition comprising an isolated polypeptide that binds specifically to at least a portion of an outer membrane protein selected from the group consisting of OmpA, PAL, and MLP, in an amount effective to enhance clearance of at least one sepsis-related soluble factor released by Gram-negative bacteria into blood of the subject.

In one embodiment the soluble factor is LPS. In another embodiment the soluble factor is OmpA. In a further embodiment the soluble factor is PAL. In yet another embodiment the soluble factor is MLP.

The invention will be more fully understood by reference to the following figures and detailed description.

### BRIEF DESCRIPTION OF THE FIGURES

FIG. 1 depicts an immunoblot (Milliblot) analysis of monoclonal antibodies using lysates of mid-log phase E. coli O6 bacteria as antigen. Primary antibodies for the immunoblots include polyclonal mouse anti-J5 IgG (lane 1) and monoclonal antibodies 2D3 (lane 2), 6D7 (lane 3), and 1C7 (lane 4). Estimated molecular weights of the bands (kDa) are indicated at the left.

FIG. 2 depicts an immunoblot analysis of OmpA-deficient bacteria. Mid-log phase bacteria are electrophoresed on 16% SDS-polyacrylamide gels and transferred to nitrocellulose. Staining antibodies include polyclonal rabbit anti-J5 IgG (left panel), and a monoclonal antibody directed to the 35 kDa OMP (2D3, right panel). Bacterial strains are: wild type OmpA⁺ E. coli O18:K1:H7 (lane 1); E91, an OmpA-deleted mutant of E. coliO18:K1:H7 (lane 2); E69, and an OmpA-restored mutant of E. coli O18:K1:H7 (lane 3). Molecular weight markers (kDa) are as at the left.

FIG. 3 depicts an immunoblot analysis of recombinant OmpA. Primary antibodies include polyclonal mouse anti-J5 IgG (left panel) and the monoclonal antibody directed against the 35 kDa OMP (2D3, right panel).

FIG. 4 depicts an immunoblot analysis of PAL-deficient bacteria. Staining antibodies include polyclonal rabbit anti-J5 IgG (left panel), and monoclonal antibody 6D7 (right panel). Bacterial strains are: E. coli K12 p400 containing PAL (lane 1); CH202, a PAL-deficient mutant of E. coli K12 p400 (lane 2); CH202 prC2, a PAL-restored mutant of CH202 (lane 3); E. coli K12 1292 containing PAL (lane 4); JC7752, a PAL-deficient mutant of 1292 (lane 5); and JC7752 p417, a PAL-restored mutant of JC7752 (lane 6).

FIG. 5 depicts an immunoblot analysis of MLP-deficient bacteria. Staining antibodies include polyclonal rabbit anti-J5 IgG (left panel), and monoclonal antibody 1C7 (right panel). Bacterial strains are: E. coli O18K⁺ (lane 1); E. coli K12 JE5505, an MLP-deficient mutant of E. coli K12 (lane 2); and E. coli K12AT1360, a closely related isolate of E. coli K12 containing MLP (lane 3).

FIG. 6 depicts an immunoblot analysis of OMP-containing samples released into human serum. Eluted samples were stained with murine monoclonal IgGs directed against OmpA (2D3), PAL (6D7), and MLP (1C7) (left three panels), and with polyclonal mouse anti-J5 IgG and a murine monoclonal IgG directed to the O-polysaccharide chain of E. coli O18 LPS (right two panels). Samples for each panel were affinity-purified with: rabbit anti-J5 IgG (lane 1), rabbit O-chain specific anti-LPS IgG (lane 2), and normal rabbit IgG (lane 3). Molecular weight markers are as indicated.

FIG. 7 depicts an immunoblot analysis of bacterial fragments released into the blood of burned rats with E. coli O18K⁺ sepsis. Blots obtained from two representative rats are shown. Lanes correspond to samples from affinity purified plasma collected from bacteremic rats prior to (lane 1) and 3 hours after (lanes 2, 3, 4) intravenous administration of ceftazidime. Antigens were eluted from polyclonal rabbit anti-J5 IgG (lanes 1 and 2), normal rabbit IgG (lane 3), and polyclonal rabbit IgG directed against the O-polysaccharide side chain of E. coli O18 LPS (lane 4) and developed with a mixture of monoclonal antibodies directed against each of the three OMPs (2D3, 6D7, and 1C7). Black arrows to the right of the blots indicate the 5-9 kDa, 18 kDa, and 35 kDa OMPs. White arrows to right of the figure indicate cross-reactive IgG bands (amplified by the more sensitive chemiluminescence technique). Molecular weight markers (kDa) are at the left.

### DETAILED DESCRIPTION

The present invention relates to three outer membrane proteins released by Gram-negative bacteria when the latter are incubated in human serum. The same outer membrane proteins are released into the circulation in an experimental model of sepsis, and they are bound by IgG in the cross-protective antiserum raised to Escherichia coli J5 (J5 antiserum). It has now been discovered that the identities of the three outer membrane proteins are outer membrane protein A (OmpA), peptidoglycan-associated lipoprotein (PAL), and murein lipoprotein (MLP).

OmpA was initially described by Henning and coworkers in 1975. Hindennach I and Henning U, Eur J Biochem 59:207-213(1975); Garten W et al., Eur J Biochem 59:215-221 (1975). It has 325 amino acid residues and exhibits heat-modifiable electrophoretic mobility on SDS-PAGE. Chen R et al., Proc Natl Acad Sci USA 77:4592-4596 (1980); Nakamura K and Mizushima S, J Biochem 80:1411-1422 (1976). The N-terminal domain of OmpA is comprised of 177 amino acids and is believed to traverse the outer membrane eight times. Klose M et al., J Biol Chem 268:25664-25670 (1993). The C-terminal domain is believed to protrude into the periplasmic space. OmpA is involved in maintaining the shape of bacteria, serves as a phage receptor and a receptor for F-mediated conjugation, and has limited pore-forming properties. Sonntag I et al., J Bacteriol 136:280-285 (1978); Sugawara E and Nikaido H, J Biol Chem 267:2507-2511 (1992); Sugawara E and Nikaido H, J Biol Chem 269:17981-17987 (1994). OmpA enhances uptake of LPS into macrophages and has been reported to be involved in E. coli invasion of the central nervous system. Korn A et al., Infect Immun 63:2697-2705 (1995); Prasadarao NV et al., Infect Immun 64:146-153 (1996). An OmpA-deficient mutant of the virulent bacterial strain, E. coli O18K1 was shown to be less virulent that its OmpA+parent strain in neonatal rat and embryonated chick egg models of sepsis. Weiser J N and Gotschlich E C, Infect Immun 59:2252-2258 (1991).

PAL was initially characterized and described by Mizuno. Mizuno T, J Biochem 89:1039-1049 (1981). It has 173 amino acid residues and is closely, but not covalently, associated with the peptidoglycan layer. Lazzaroni J-C and Portalier R, Mol Microbiol 6:735-742 (1992); Mizuno T, J Biochem 89:1039-1049 (1981); Mizuno T, J Biochem 86:991-1000 (1979). PAL has a hydrophobic region of 22 amino acids at the N-terminal domain that interacts with the outer membrane. Lazzaroni J-C and Portalier R, Mol Microbiol 6:735-742 (1992). The C-terminal domain is involved in interactions with the peptidoglycan layer. Lazzaroni J-C and Portalier R, Mol Microbiol 6:735-742 (1992).

MLP was first described and characterized by Braun. Hantke K and Braun V, Eur J Biochem 34:284-296 (1973); Braun V and Wolff H, Eur J Biochem 14:387-391 (1970); Braun V and Bosch V, Eur J Biochem 28:51-69 (1972). It is the most abundant outer membrane protein. Braun V and Wolff H, Eur J Biochem 14:387-391(1970). MLP has 58 amino acid residues and exists in two forms, a free form and a form that is covalently linked to peptidoglycan by the C-terminal domain. Braun V and Bosch V, Eur J Biochem 28:51-69 (1972); Braun V, Biochim Biophys Acta 415:335-377 (1975). Recently Zhang reported that MLP induces lethal shock in a strain of mouse (C3H/HeJ) that is genetically hyporesponsive to LPS. Zhang H et al., J Immunol 159:4868-4878 (1997). Furthermore, they found that MLP was synergistic with LPS for lethal toxicity.

Applicants previously have shown that epitopes of three proteins are exposed on the surface of bacteria that have been incubated in human serum, and that antiserum raised to a rough mutant vaccine of E. coli J5 results in high titers of antibodies that bind to the same three proteins on the bacterial surface. The identity of two of these proteins as PAL and MLP is surprising, as both proteins are situated in the deep periplasmic space and only short N-terminal segments are believed to interact with the outer membrane. Lazzaroni J -C and Portalier R, Mol Microbiol 6:735-742 (1992); Steinemann S et al., Arterioscler Thromb 14:1202-1209 (1994). Therefore, the increased clearance of heterologous smooth bacterial strains by infusion of antiserum to E. coli J5 (Sakulramrung R and Dominigue G. J, J Infect Dis 151:995-1004 (1985)) may be mediated by binding of immunoglobulin in this antiserum to epitopes of OmpA, PAL, and MLP on the bacterial surface.

Circulating bacterial toxins are believed to be important in the pathogenesis of Gram-negative sepsis, but little is actually known about the composition of released bacterial components. Most studies have focused on release of LPS, and it has been assumed that LPS is released in membrane blebs that then disaggregate into LPS monomers. Tesh V L et al., J Immunol 137:1329-1335 (1986); Tesh V L and Morrison D C, J Immunol 141:3523-3531 (1988); Danner R L et al., Chest 99:169-175 (1991); Pearson F C et al., J Clin Microbiol 21:865-868 (1985); Winchurch R A et al., Surgery 102:808-812 (1987); Wessels B C et al., Crit Care Med 16:601-605 (1988); Brandtzaeg P et al., Regul Pept 24:37-44 (1989); van Deventer S J et al., Lancet 1:605-609 (1988); Natanson C et al., J Clin Invest 83:243-251 (1989); Shenep J L et al., J Infect Dis 157:565-568 (1988); Munford R S et al., J Clin Invest 70:877-888 (1982). Prior studies have shown that live bacteria incubated in human serum release fragments containing OMPs and LPS (OMP/LPS complexes) that can be affinity-purified using antibodies directed to the O-polysaccharide side chain of LPS. Hellman J et al., J Infect Dis 176:1260-1268 (1997); Freudenberg M A et al., Microb Pathog 10:93-104 (1991). Freudenberg reported that samples that were affinity-purified from filtrates of serum-exposed Salmonella abortus equi bacteria using anti-LPS IgG also contained OmpA and a second protein of MW 17 kDa that was not identified. Freudenberg M A et al., Microb Pathog 10:93-104 (1991).

Applicants now have found that OMP/LPS complexes that contain at least three OMPs are released in vivo into the bloodstream in an infected bum model of Gram-negative sepsis. The 18 kDa OMP is also released into septic rat blood in a form that is separate from the OMP/LPS complexes and is selectively affinity purified by IgG in antiserum raised to heat-killed E. coli J5 bacteria.

Although many studies report that proteins that are tightly associated with LPS are biologically active, the role of OMPs in the pathogenesis of sepsis has not been defined. Melchers F et al., J Exp Med 142:473-482 (1975); Doe W F et al., J Exp Med 148:557-568 (1978); Goodman G W and Sultzer B M, J Immunol 122:1329-1334 (1979); Goodman G W and Sultzer B M, Infect Immun 24:685-696 (1979); Chen Y et al., Infect Immun 28:178-184 (1980); Goldman R C et al., J Immunol 127:1290-1294 (1981); Galdiero F et al., Infect Immun 46:559-563 (1984); Bjornson B H et al., Infect Immun 56:1602-1607 (1988); Johns M A et al., Infect Immun 56:1593-1601 (1988); Hauschildt S et al., Eur J Immunol 20:63-68 (1990); Porat R et al., Infect Immun 60:1756-1760 (1992); Mangan D F et al., Infect Immun 60:1684-1686 (1992); Galdiero F et al., Infect Immun 61:155-161 (1993); Manthey C L et al., J Immunol 153:2653-2663 (1994); Snapper C M et al., J Immunol 155:5582-5589 (1995); Korn A et al., Infect Immun 63:2697-2705 (1995); Zhang H et al., J Immunol 159:4868-4878 (1997); Giambartolomei G H et al., Infect Immun 67:140-147 (1999). Given these studies and the previously described protective efficacy of J5 antiserum, it appears that OMPs play a role in the pathogenesis of Gram-negative sepsis.

The invention in one aspect provides vaccine compositions that incorporate an effective amount of at least one isolated outer membrane protein selected from OmpA, PAL, MLP, and any immunogenic portion thereof, prepared in a pharmaceutically suitable carrier.

In a related aspect, the invention provides a method of making a vaccine composition, involving placing an effective amount of at least one isolated outer membrane protein selected from OmpA, PAL, MLP, and any immunogenic portion thereof, in a pharmaceutically suitable carrier.

The term “effective amount” as used herein refers to the amount necessary or sufficient to realize a desired biologic effect. For example, an effective amount of an isolated outer membrane protein in a vaccine composition is that amount necessary to cause the development of an antigen-specific immune response upon exposure to the OMP, thus inducing protection. The effective amount for any particular application can vary depending on such factors as the particular OMP being administered, the particular adjuvant (if any) used in conjunction with the antigen, the route of administration, the size of the subject, the competence of the immune system of the subject, or the severity of the disease or condition. One of ordinary skill in the art can empirically determine the effective amount of a particular OMP antigen without necessitating undue experimentation.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the vaccine composition or pharmaceutical composition can be administered to a subject by any mode allowing the OMP antigen to be taken up by the appropriate target cells. “Administering” the vaccine or pharmaceutical composition of the present invention may be accomplished by any means known to the skilled artisan. Preferred routes of administration include but are not limited to oral, transdermal (e.g. via a patch), parenteral injection (subcutaneous, intradermal, intravenous, intramuscular, intraperitoneal, intrathecal, etc.), or mucosal (intranasal, intratracheal, inhalation, and intrarectal, intravaginal etc). An injection may be in a bolus or a continuous infusion.

For example the vaccine and pharmaceutical compositions according to the invention are often administered by intramuscular or intradermal injection, or other parenteral means, or by biolistic “gene-gun” application to the epidermis. They may also be administered by intranasal application, inhalation, topically, intravenously, orally, or as implants, and even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also can include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:1527-1533 (1990), which is incorporated herein by reference.

The pharmaceutical compositions are preferably prepared and administered in dose units. Liquid dose units are vials or ampoules for injection or other parenteral administration. Solid dose units are tablets, capsules and suppositories. For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units. Multiple administration of doses at specific intervals of weeks or months apart is usual for boosting the antigen-specific responses.

The antigens and adjuvants may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of an antigen optionally included in a pharmaceutically suitable carrier. The term “pharmaceutically suitable carrier” means one or more compatible solid or liquid filler, diluants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

Compositions suitable for parenteral administration conveniently comprise sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose any bland fixed mineral or non-mineral oil may be employed including synthetic mono- or di-glycerides. In addition, fatty acids such as oleic acid find use in the preparation of injectables. Carrier formulations suitable for subcutaneous, intramuscular, intraperitoneal, intravenous, etc. administrations may be found in Remington's Pharmaceutical Sciences, Mack Publishing Company, Easton, Pa.

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer-based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

As used herein in reference to an OMP or other polypeptide, the term “isolated” means separated from its native environment in sufficiently pure form so that it can be manipulated or used for any one of the purposes of the invention. An isolated compound refers to a compound which represents at least 10 percent of the compound present in the mixture and exhibits a detectable (i.e., statistically significant) biological activity when tested in conventional biological assays such as those described herein. Preferably the isolated compound represents at least 50 percent of the mixture; more preferably at least 80 percent of the mixture; and most preferably at least 90 percent or at least 95 percent of the mixture. Thus, isolated means sufficiently pure to be used (i) to raise and/or isolate antibodies, (ii) as a reagent in an assay, or (iii) for sequencing, etc.

Thus, in the preferred embodiments, the isolated outer membrane proteins are immunogenic and can be used to generate binding polypeptides (e.g., antibodies) for use in diagnostic and therapeutic applications. Such binding polypeptides also are useful for detecting the presence, absence, and/or amounts of particular OMPs in a sample such as a biological fluid or biopsy sample.

The invention also provides isolated OMPs (including whole proteins and partial proteins), encoded by previously known nucleic acids. Outer membrane proteins can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including antigenic peptides (such as are presented by MHC molecules on the surface of a cell for immune recognition) also can be synthesized chemically using well-established methods of peptide synthesis.

The term “outer membrane protein” as used herein in reference to the three specific OMPs OmpA, PAL, and MLP shall include both the polypeptide component alone and the polypeptide component in association with lipid. In this way, the term “outer membrane protein” can encompass the fact that PAL and MLP occur naturally as lipoproteins. The association between the polypeptide component and lipid can be covalent or non-covalent.

The term “OmpA” as used herein refers to any of a number of immunologically cross-reactive cell wall polypeptide components from heterologous Gram-negative bacteria known in the art as outer membrane protein A or OmpA. As used herein, OmpA is distinct from LPS and exemplified by, but not limited to, OmpA of E. coli K12, GenBank accession no. P02934. It is recognized that OmpA can be released into human serum in vitro and in vivo in complexes that also contain LPS. The term “OmpA” as used herein shall include both the polypeptide component alone and the polypeptide component in association with lipid.

The term “PAL” as used herein refers to any of a number of immunologically cross-reactive lipoprotein cell wall components from heterologous Gram-negative bacteria known in the art as peptidoglycan-associated lipoprotein or PAL. As used herein, PAL is distinct from LPS and exemplified by, but not limited to, PAL of E. coli K12, GenBank accession no. P07176. It is recognized that PAL can be released into human serum in vitro and in vivo in complexes that also contain LPS. The term “PAL” as used shall include both the polypeptide component alone and the polypeptide component in association with lipid.

The term “MLP” as used herein refers to any of a number of immunologically cross-reactive lipoprotein cell wall components from heterologous Gram-negative bacteria known in the art simply as lipoprotein, or as Braun's lipoprotein, murein lipoprotein, or MLP. As used herein, MLP is distinct from LPS and exemplified by, but not limited to, MLP of E. coli K12, GenBank accession no. P02937. It is recognized that MLP can be released into human serum in vitro and in vivo in complexes that also contain LPS. The term “MLP” as used shall include both the polypeptide component alone and the polypeptide component in association with lipid.

An “immunogenic portion” as used herein refers to any fragment of an isolated OMP that can, under appropriate conditions, induce an immune response. For an immune response involving antibodies, an immunogenic portion will include an antigenic determinant which is the target of antibody binding. With respect to proteins and polypeptides, antigenic determinants involve specific amino acid residues in a particular three-dimensional conformation. These amino acid residues must be exposed on the surface of the protein or polypeptide in order to be immunogenic. For an immune response involving T cells, an immunogenic portion of a protein or polypeptide is most often an immunodominant determinant or, alternatively, a cryptic determinant. Sercarz EE et al., Annu Rev Immunol 11:729-766 (1993). T-cell response to both these types of determinants involve antigen processing, i.e., intracellular partial degradation of protein or polypeptide into short oligopeptides which are subsequently associated with major histocompatibility complex (MHC) molecules and presented on the surface of the T cell.

An “adjuvant” is any molecule or compound which can stimulate or augment the stimulation of a humoral and/or cellular immune response. An adjuvant typically is administered in association with exposure to an antigen to enhance the immune response to the antigen. An immune system stimulant exerts a mitogenic effect on immune system cells and can cause increased cytokine expression by vertebrate lymphocytes. A number of adjuvants are well known in the art. These can include, for instance, adjuvants that create a depot effect, immune-stimulating adjuvants, adjuvants that create a depot effect and stimulate the immune system, and mucosal adjuvants.

An adjuvant that creates a depot effect is an adjuvant that causes an antigen to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

An immune-stimulating adjuvant is an adjuvant that causes direct activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland); Leishmania elongation factor (a purified Leishmania protein; Corixa Corporation, Seattle, Wash.); and CpG DNA (WO 96/02555).

Adjuvants that create a depot effect and stimulate the immune system are those compounds which have both of the above-identified functions. This class of adjuvants includes but is not limited to ISCOMS (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21; SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (which contains a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

A mucosal adjuvant is an that is capable of inducing a mucosal immune response in a subject when administered to a mucosal surface in conjunction with an antigen. Mucosal adjuvants include but are not limited to bacterial toxins: e.g., Cholera toxin (CT); CT derivatives including but not limited to CT B subunit (CTB) (Wu et al., 1998, Tochikubo et al., 1998); CTD53 (Val to Asp) (Fontana et al., 1995); CTK97 (Val to Lys) (Fontana et al., 1995); CTK104 (Tyr to Lys) (Fontana et al., 1995); CTD53/K63 (Val to Asp, Ser to Lys) (Fontana et al., 1995); CTH54 (Arg to His) (Fontana et al., 1995); CTN107 (His to Asn) (Fontana et al., 1995); CTE114 (Ser to Glu) (Fontana et al., 1995); CTE112K (Glu to Lys) (Yamamoto et al., 1997a); CTS61F (Ser to Phe) (Yamamoto et al., 1997a, 1997b); CTS106 (Pro to Lys) (Douce et al., 1997, Fontana et al., 1995); and CTK63 (Ser to Lys) (Douce et al., 1997, Fontana et al., 1995); zonula occludens toxin (zot); Escherichia coli heat-labile enterotoxin (Labile Toxin, LT); LT derivatives including but not limited to LT B subunit (LTB) (Verweij et al., 1998); LT7K (Arg to Lys) (Komase et al., 1998; Douce et al., 1995); LT61F (Ser to Phe) (Komase et al., 1998); LT112K (Glu to Lys) (Komase et al., 1998); LT118E (Gly to Glu) (Komase et al., 1998); LT146E (Arg to Glu) (Komase et al., 1998); LT192G (Arg to Gly) (Komase et al., 1998); LTK63 (Ser to Lys) (Marchetti et al., 1998; Douce et al., 1997, 1998; Di Tommaso et al., 1996); and LTR72 (Ala to Arg) (Giuliani et al., 1998); Pertussis toxin (PT) (Lycke et al., 1992; Spangler BD, 1992; Freytag and Clemments, 1999; Roberts et al., 1995; Wilson et al., 1995) including PT-9K/129G (Roberts et al., 1995; Cropley et al., 1995); toxin derivatives (Holmgren et al., 1993; Verweij et al., 1998; Rappuoli et al., 1995; Freytag and Clements, 1999); lipid A derivatives (e.g., monophosphoryl lipid A, MPL) (Sasaki et al., 1998; Vancott et al., 1998); muramyl dipeptide (MDP) derivatives (Fukushima et al., 1996; Ogawa et al., 1989; Michalek et al., 1983; Morisaki et al., 1983); bacterial outer membrane proteins (e.g., outer surface protein A (OspA); lipoprotein of Borrelia burgdorferi; outer membrane protein of Neisseria meningitidis) (Marinaro et al., 1999; Van de Verg et al., 1996); oil-in-water emulsions (e.g., MF59) (Barchfield et al., 1999; Verschoor et al., 1999; O'Hagan, 1998); aluminum salts (Isaka et al., 1998, 1999); and saponins (e.g., QS21) (Aquila Biopharmaceuticals, Inc., Worcester, Mass.) (Sasaki et al., 1998; MacNeal et al., 1998), ISCOMS; MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micelle-forming agent; IDEC Pharmaceuticals Corporation, San Diego, Calif.); Syntext Adjuvant Formulation (SAF; Syntex Chemicals, Inc., Boulder, Colo.); poly[di(carboxylatophenoxy)phosphazene (PCPP polymer; Virus Research Institute, USA); and Leishmania elongation factor (Corixa Corporation, Seattle, Wash.).

The invention in another aspect provides an adjuvant that includes an effective amount of at least one isolated outer membrane protein selected from OmpA, PAL, MLP, and any combination thereof. It is believed that these compounds are useful as adjuvants themselves. It is well known in the art that various killed whole bacteria in addition to killed M. tuberculosis are useful as adjuvants. LPS itself is a powerful adjuvant, but its utility is severely restricted by its very significant toxicity. Since isolated outer membrane proteins appear to have biologic activity separate from LPS, and because LPS preparations commonly contain at least some outer membrane proteins, it is believed that these outer membrane proteins themselves have adjuvant activity.

In yet another aspect the invention provides pharmaceutical compositions useful for treating a subject infected with Gram-negative bacteria. Such pharmaceutical compositions include an isolated polypeptide that binds specifically to at least a portion of OmpA, PAL, or MLP, prepared in a pharmaceutically suitable carrier. The binding interaction between the pharmaceutical composition and the outer membrane protein will typically but not necessarily involve a non-covalent association between them. The effect of the specific binding in vivo can result in passive immunization. Mechanisms by which such passive immunization is believed to exert an effect are disclosed below. The effect of the specific binding in vivo and in vitro can also lead to functional deactivation of the outer membrane protein by, for example, sequestering or otherwise making inaccessible a biologically active site on the OMP. The types of pharmaceutical compositions contemplated in this aspect of the invention include monoclonal antibodies, fragments of monoclonal antibodies, agents formed in part by monoclonal antibodies or fragments thereof, polyclonal antibodies, and synthetic polypeptides that may be generated as part of a combinatorial library of such polypeptides.

In a related aspect, the invention further provides a method of making a pharmaceutical compositions useful for treating a subject infected with Gram-negative bacteria. The method involves placing an effective amount of at least one isolated polypeptide that binds selectively to at least a portion of an outer membrane protein selected from OmpA, PAL, MLP, in a pharmaceutically suitable carrier.

As used herein, the term “subject” refers to a vertebrate. In certain embodiments the subject is a human.

A “subject infected with Gram-negative bacteria” refers to a subject in which living Gram-negative bacteria have breached normal anatomic and functional protective barriers (e.g., skin, mucosa, etc.) and survived to multiply in a tissue, fluid, or space within the subject that is normally sterile. Typically, but not necessarily, Gram-negative bacteria can be cultured from infected tissue or body fluid obtained from a subject infected with Gram-negative bacteria. A “subject infected with Gram-negative bacteria” may have, but need not have, Gram-negative sepis.

As used herein, the term “Gram-negative bacteria” refers to bacteria that are known in the art as members of the Enterobacteriaceae, non-enteric Gram-negative bacteria, and anaerobic Gram-negative bacteria. These include but are not limited to the following:

Enterobacteriaceae—Buttiauxella spp., Cedeca spp., Cedecea spp., Citrobacter spp., Edwardsiella spp., Enterobacter spp., Escherichia spp., Ewingella spp., Hafnia spp., Klebsiella spp., Kluyvera spp., Leclercia spp., Leminorell spp., Moellerella spp., Morganella spp., Obesumbacterium spp., Proteus spp., Providencia spp., Rhanella spp., Salmonella spp., Serratia spp., Shigella spp., Trabulsiella spp., Tutamella spp., Xenorhabdus spp., Yersinia spp., Yokenella spp., (and various “enteric groups” that are not as yet assigned).

Non-enteric Gram-negative bacteria—Acinetobacter spp., Achromobacter spp., Actinobacillus spp., Aeromonas spp., Alcaligenes spp., Arcobacter spp., Bordetella spp., Borrelia spp., Branhamella spp., Brucella spp., Campylobacter spp., Capnocytophaga spp., Cardiobacterium spp., Chromobacterium spp., Commamonas spp., Eikenella spp., Flavimonas spp., Francisella spp., Haemophilus spp., Helicobacter spp., Kingella spp., Legionella spp., Moraxella spp., Neisseria spp., Ochrobactrum spp., Oligella spp., Pasteruella spp., Plesiomonas spp., Protomonas spp., Pseudomonas spp., Sphingobacterium spp., Streptobacillus spp., Vibrio spp., Weeksell spp., Xanthomonas spp., Yersinia spp.

Anaerobic Gram-negative bacteria—Bacteroides spp., Fusobacterium spp.

The invention also embraces isolated polypeptides capable of binding selectively to at least a portion of an OMP selected from OmpA, PAL, or MLP. Such polypeptides can include, for example, antibodies or fragments of antibodies (“binding polypeptides”). Antibodies include monoclonal and polyclonal antibodies, prepared according to conventional methodology. See, e.g., Harlow & Lane, “Antibodies: A Laboratory Manual,” Cold Spring Harbor Laboratory, 1988.

The term “antibody” as used herein means at least a portion of an immunoglobulin molecule (see W. E. Paul, ed., “Fundamental Immunology,” Lippincott-Raven, Philadelphia, 1999, pp. 37-74) capable of binding to an antigen. Preferably the antibody belongs to the immunoglobulin G (IgG) class of antibodies. According to this definition, the term “antibody” includes not only intact antibodies but also various forms of modified or altered antibodies, such as an Fv fragment containing only the light and heavy chain variable regions, an Fab or (Fab)′₂ fragment containing the variable regions and parts of the constant regions, a single-chain antibody, and the like. Bird et al., Science 242:424-426 (1988); Huston et al., Proc Natl Acad Sci USA 85:5879-5883 (1988). The antibody may be of animal (especially mouse or rat) or human origin or may be chimeric (Morrison S et al., Proc Natl Acad Sci USA 81:6851-6855 (1984)) or humanized (Jones et al., Nature 321:522-525 (1986), and published UK patent application 8707252). Methods of producing antibodies suitable for use in the present invention are well known to those skilled in the art and can be found described in such publications as Harlow & Lane, “Antibodies: A Laboratory Manual,” Cold Spring Harbor Laboratory, 1988. The genes encoding the antibody chains may be cloned in cDNA genomic form by any cloning procedure known to those skilled in the art. See for example Maniatis et al., “Molecular Cloning: A Laboratory Manual,” Cold Spring Harbor Laboratory, 1982.

In another embodiment a pharmaceutical composition for use in treating a subject infected with Gram negative bacteria can include an isolated polyclonal antibody that binds specifically to at least a portion of OmpA, PAL, or MLP, prepared in a pharmaceutically suitable carrier. The binding interaction and the effects of such binding between the pharmaceutical composition and the outer membrane protein will be as just described above in reference to an isolated polypeptide that binds specifically to at least a portion of OmpA, PAL, or MLP.

According to this aspect of the invention, the polyclonal antibody binds to OmpA, PAL, MLP, or any combination of these OMPs, but not to at least one other component bound by J5 antiserum. For example, the polyclonal antibody of the invention can in some embodiments bind to OmpA, PAL, or MLP, but not to other LPS-associated lipoproteins.

In this aspect of the invention, the polyclonal antibody is raised by immunizing an animal, preferably a mammal, with an effective amount of isolated OmpA, PAL, MLP, or any combination of these OMPs. The polyclonal antibody so prepared differs from J5 antiserum insofar as the latter is raised against heat-killed whole bacteria and thus binds to antigens in addition to those related only to OmpA, PAL, and MLP.

In a particular embodiment of this aspect of the invention, the polyclonal antibody can be raised by immunizing a human with an effective amount of isolated OmpA, PAL, MLP, or any combination of these OMPs. The resulting human antiserum can be used effectively in human subjects.

Binding polypeptides that bind selectively to certain OMPs also may be derived from sources other than antibody technology. For example, such polypeptide binding agents can be provided by degenerate peptide libraries which can be readily prepared in solution, in immobilized form, as bacterial flagella peptide display libraries or as phage display libraries. Combinatorial libraries also can be synthesized of peptides containing one or more amino acids. Libraries further can be synthesized of peptides and non-peptide synthetic moieties.

Phage display can be particularly effective in identifying binding peptides useful according to the invention. Briefly, one prepares a phage library (using, e.g., m 13, fd, or lambda phage), displaying inserts from 4 to about 80 amino acid residues using conventional procedures. The inserts may represent, for example, a completely degenerate or biased array. One then can select phage-bearing inserts which bind to the OMP or a complex containing an OMP. This process can be repeated through several cycles of reselection of phage that bind to the OMP or complex. Repeated rounds lead to enrichment of phage bearing particular sequences. DNA sequence analysis can be conducted to identify the sequences of the expressed polypeptides. The minimal linear portion of the sequence that binds to the OMP or complex can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the OMPs. Thus, the OMPs of the invention, or a fragment thereof, or complexes of OMP can be used to screen peptide libraries, including phage display libraries, to identify and select peptide binding polypeptides that selectively bind to the OMPs of the invention. Such molecules can be used, as described, for screening assays, for purification protocols, for interfering directly with the functioning of OMPs and for other purposes that will be apparent to those of ordinary skill in the art.

OmpA, PAL, MLP, or a fragment thereof, also can be used to isolate naturally occurring polypeptide binding partners which may associate with the OMPs in vitro or in vivo. Recently it has come to be appreciated that certain Toll-like receptors (TLRs) are responsible for cellular response to microbial products, including LPS and lipoproteins. Hirschfeld M et al., J Immunol 165:618-622 (2000). TLR4 and TLR2 have been associated with LPS signaling, and a point mutation in the tlr4 gene in C3H/HeJ mice has been reported to account for the observed hyporesponsiveness of that strain to LPS. Thus OmpA, PAL, and MLP may be useful, for example, in further elucidating the details of TLR-mediated signaling as well as other receptors and pathways involved in LPS signaling. Isolation of binding partners may be performed according to well-known methods. For example, isolated OmpA, PAL, or MLP can be attached to a substrate, and then a solution suspected of containing an OMP-binding partner may be applied to the substrate. If the binding partner for OmpA, PAL, or MLP is present in the solution, then it will bind to the substrate-bound OMP. The binding partner then may be isolated for identification and further study. Other proteins which are binding partners for OmpA, PAL, or MLP, may be isolated by similar methods without undue experimentation.

The invention in another aspect provides an immortal cell line which secretes a polypeptide that binds specifically to OmpA, PAL, MLP, or immunogenic portions thereof. Preferably, the secreted polypeptide is a monoclonal antibody directed against OmpA, PAL, or MLP. In alternative embodiments the secreted polypeptide can also be a fragment of a monoclonal antibody directed against OmpA, PAL, or MLP, or it can be a fusion protein incorporating an antigen-binding portion of such an antibody.

As used herein, “immortal cell line” refers to a hybridoma, myeloma, or a transfected cell line that, under proper conditions, can be propagated indefinitely. In a preferred embodiment the immortal cell line is a hybridoma prepared by cell fusion between splenocytes from an immunized animal and a myeloma according to standard techniques. Kohler G et al., Eur J Immunol 6:292-295 (1976). In another embodiment the immortal cell line can be a myeloma or non-immune cell that is transfected with a nucleic acid that operably encodes an antibody, antibody fragment, fusion protein, or the like. In yet another embodiment the immortal cell line can be a myeloma or hybridoma that is directed to express a desired polypeptide through homologous recombination.

The term “secretes” as used herein refers to expression of polypeptide in a form that can be isolated for the purposes of the invention. In the instance of a hybridoma, the polypeptide typically is expressed and released into the medium in which the hybridoma is grown. Forms of expression that result in polypeptides that remain associated with the cell membrane or that remain in an intracellular compartment are also encompassed by the use of this term.

In yet another aspect he invention further provides a method of actively immunizing a subject against infection due to Gram-negative bacteria. The method involves administering to a subject an isolated OMP antigen selected from OmpA, PAL, MLP, or an immunogenic portion thereof, prepared in a pharmaceutically suitable carrier, in an amount effective for inducing protection of the subject against infection due to Gram-negative bacteria. The method can entail immunization against any one or any combination of the three OMP antigens, and it can further entail administration of the OMP antigen with an adjuvant that is distinct from OmpA, PAL, or MLP. Examples of such adjuvants are listed above. In this context, an effective amount is that amount sufficient to induce a protective immune response to the antigen. This can be manifest as a titer of circulating IgG antibody specific for the antigen which is at least about 1:16 or at least twice that of a control titer as measured in an unexposed nonimmune subject. Alternatively, it can be manifest as a prompt anamnestic response (with increase in antigen-specific IgG titer) upon reexposure to the antigen.

The term “antigen” broadly includes any type of molecule, typically a polypeptide or polysaccharide, which is recognized by a host immune system as being foreign. An “OMP antigen” as used herein refers to any intact form or immunogenic fragment of OmpA, PAL, or MLP that can induce a immune response specific to that OMP. A specific immune response typically involves the generation of antibodies that bind specifically to at least one epitope of the antigen. A specific immune response can also involve the response by T cells bearing antigen receptors that specifically recognize peptide fragments of an antigen in association with major histocompatibility complex (MHC). Thus a specific immune response to an OMP antigen can include the generation of antibodies that bind specifically to at least one epitope of the OMP antigen and the response by T cells bearing antigen receptors that specifically recognize peptide fragments of an OMP antigen in association with MHC.

The invention in another aspect provides a method of treating a subject who has an infection with Gram-negative bacteria. The method involves administering to a subject in need of such treatment an isolated polypeptide that binds specifically to OmpA, PAL, or MLP in an amount effective to treat the infection with the Gram-negative bacteria. Preferably the isolated polypeptide is administered in an amount effective to inhibit growth of the Gram-negative bacteria in vivo. This inhibitory effect on growth can be determined by methods well known in the art, including, e.g., comparing the number of colony-forming units in a standard culture taken from an infected body fluid in the presence of and in the absence of the polypeptide. An inhibitory effect due to the presence of the polypeptide would be associated with a diminished or declining number of colonies in comparison to the corresponding number of colonies in the absence of the polypeptide. More preferably the isolated polypeptide is administered in an amount effective to inhibit Gram-negative sepsis. The isolated polypeptide can be an antibody or another polypeptide (as described above), so long as it binds specifically to OmpA, PAL, or MLP in vivo. The isolated polypeptide is administered in a pharmaceutically suitable carrier.

The term “treating” is defined as administering, to a subject, a therapeutically effective amount of a compound that is sufficient to prevent the onset of, alleviate the symptoms of, or stop the progression of a disorder or disease being treated. The phrase “therapeutically effective amount” means that amount of a compound which prevents the onset of, alleviates the symptoms of, or stops the progression of a disorder or disease being treated. Thus, as used herein, an amount effective to treat an infection caused by Gram-negative bacteria is an amount effective to prevent the onset of, alleviate the symptoms of, or stop the progression of an infection caused by Gram-negative bacteria.

As used herein, the term “inhibit Gram-negative sepsis” refers to inhibition of any aspect of the multitude of inducing and responding signals and events which are associated with the systemic inflammatory response to infection with Gram-negative bacteria. This is meant to encompass both early and late sepsis, i.e., both before and during the stage with cardiovascular decompensation and end organ dysfunction and injury. Early events and signals in the development of sepsis can include induction of proinflammatory cytokines, e.g., IL-1β, IL-6, and TNF-α, as well as elaboration and release of other cytokines and mediators, including IL-8, gamma interferon (IFN-γ), chemokines, migration inhibitory factor (MIF), nitric oxide, kinins, complement, platelet activating factor (PAF), etc. Organs particularly susceptible to sepsis-related dysfunction and injury in late sepsis include lung, liver, and kidneys. Other problems frequently encountered in late sepsis include dysfunction of the skin, gastrointestinal tract, central nervous system, bone marrow, and cardiovascular system.

Without meaning to be bound by any particular theory, the mechanisms by which the inhibition of Gram-negative sepsis is believed to be achieved include clearance, neutralization, and opsonization. These various mechanisms can be applied to whole bacteria, insoluble fragments of bacteria, and soluble factors released from bacteria. Soluble factors released from bacteria include the OMPs themselves, either free or in complexes with LPS.

The term “clearance” as used herein refers to removal from the circulation. This can include clearance by excretion, sequestration, degradation, and the like.

“Insoluble fragments of Gram-negative bacteria” as used herein refers to any particulate component or aggregate of components originating from Gram-negative bacteria which can be precipitated out of serum or out of solution by centrifugation. Examples of such fragments include cell wall fragments, membrane blebs, etc.

The term “neutralize” as used herein refers to the abrogation of biological activity of a molecule by steric interference of the interaction between the biologically active molecule and its cellular receptor. As applied to whole bacteria, the term “neutralize” refers to abrogation of biological activity of whole bacteria by steric interference of the interaction between the biologically active molecules on the bacteria and their receptors on cells of an infected host. Similarly, as applied to insoluble fragments of bacteria, the term “neutralize” refers to abrogation of biological activity of the fragments by steric interference of the interaction between the biologically active molecules on the fragments and their receptors on cells of an infected host.

The term “opsonize” as used herein refers to the formation of immune complexes between antibodies and their cognate antigens. Opsonization can result in phagocytosis of the bound target, elimination of the bound target from the circulation, and neutralization. In relation to whole bacteria, opsonization also can lead to cell lysis through complement activation.

According to this aspect of the invention, the method of treating a subject who has Gram-negative sepsis may further include administering to the subject an effective amount of an immune stimulant. An immune stimulant can include an adjuvant (described above), a cytokine, or a substance that induces a cytokine or costimulatory molecule.

Cytokines include interleukins, interferons, certain growth factors, and colony stimulating factors. Included among these are, e.g., interleukin (IL)-2, IL-4, IL-6, IL-10, IL-12, interferon (IFN)-γ, tumor necrosis factor (TNF)-α, transforming growth factor (TGF)-β, and granulocyte colony stimulating factor (G-CSF).

Costimulatory molecules include, for example, CD2, CD28, CD40, CD48, CD80 (B7-1), CD86 (B7-2), CD152 (CTLA-4).

Chemokines include compounds in four subfamilies based on their structure: CXC, CC, C, and CX₃C. Examples of chemokines include MIP-1α, MIP-1β, RANTES, MCP-1, MCP-2, IL-8, and GROα, among others.

Assays for immunoglobulins, cytokines, costimulatory molecules, and chemokines are well known to those skilled in the art. See, e.g., Current Protocols in Molecular Biology, John Wiley & Sons, New York, 1999. A number of commercial kits, particularly ELISAs, are available for most of these secreted products.

In yet another aspect the invention provides a method of treating a subject who has Gram-negative sepsis. The method involves administering an isolated polypeptide that binds specifically to OmpA, PAL, or MLP in an amount effective to inhibit sepsis-related release of at least one soluble factor into blood or tissue of the subject. The isolated polypeptide that binds specifically to at least a portion of OmpA, PAL, or MLP can include an antibody, a fragment of an antibody, or another polypeptide as described above.

An amount effective to inhibit sepsis-related release of at least one soluble factor into blood or tissue of the subject is an amount that, when given to a subject under conditions where the at least one soluble factor is normally released into blood or tissue in the absence of the inhibitor, is sufficient to prevent release or decrease the amount released of the at least one soluble factor in the blood or tissue in the presence of the polypeptide. Soluble factors released in relation to sepsis can include factors originating from the infective bacteria or from the host. Examples of soluble factors released from Gram-negative bacteria include OMPs, LPS, and free lipids. Examples of soluble factors of host origin include cytokines (e.g., IL-1, IL-6, TNF-α), HMG-1 (Wang H et al., Science 285:248-251 (1999)), chemokines, MIF, and nitric oxide.

The invention further provides a method of treating a subject who has Gram-negative sepsis. The method involves administering to a subject with Gram-negative sepsis an isolated polypeptide that binds specifically to at least a portion of OmpA, PAL, or MLP in an amount effective to enhance clearance of at least one sepsis-related soluble factor released by Gram-negative bacteria into blood of the subject. The isolated polypeptide that binds specifically to at least a portion of OmpA, PAL, or MLP can include an antibody, a fragment of an antibody, or another polypeptide as described above. In a preferred embodiment, the polypeptide is a monoclonal antibody specific for OmpA, PAL, or MLP. A sepsis-related soluble factor released by Gram-negative bacteria into blood of the subject can include any one or combination of the following: LPS, OmpA, PAL, and MLP.

The present invention is further illustrated by the following Examples, which in no way should be construed as further limiting. The entire contents of all of the references (including literature references, issued patents, and published patent applications) cited throughout this application are hereby expressly incorporated by reference.

### EXAMPLES

Bacterial strains, media, and growth conditions. E. coli J5 was the kind gift of J. C. Sadoff (Walter Reed Army Institute of Research, Washington, D.C.). E. coli O18:K1:H7 strain Bort (designated E. coli O18K⁺), E. coli O18:K1⁻:G2A (a nonencapsulated derivative of O18:K1:H7, designated E. coli O18K⁻), E. coli O8:K45:H1, E. coli O16:K1:H6, and E. coli O25:K5:H1 were kind gifts of A. Cross (University of Maryland Cancer Center, Baltimore). OMP-deficient E. coli K12 and E. coli O18 mutants and closely related OMP-containing bacteria were used for immunoblotting studies. E. coli O18 E91 (OmpA-deficient derivative of E. coli O18:K1:H7) and E69 (OmpA-restored derivative of E. coli O18:K1:H7) were kind gifts of K. S. Kim (Los Angeles Children's Hospital). Prasadarao NV et al., Infect Immun 64:146-153 (1996). E. coli K12 1292 (Lazzaroni J-C and Portalier R, Mol Microbiol 6:735-742 (1992)), JC7752 (PAL-deficient derivative of 1292), and 7752p417 (PAL-restored mutant of JC7752) were kindly provided by J. -C. Lazzaroni (Universite Claude Bernard, Lyon 1, France). E. coli K12 p400, CH202 (PAL-deficient mutant of p400), and CH202(pRC2) (PAL-restored derivative of CH202) were kindly provided by U. Henning (Max-Planck-Institut fir Biologie, Tübingen, Germany). Chen R and Henning U, Eur J Biochem 163:73-77 (1987).

E. coli K12 AT1360 (Lpp⁺; mutations: DE [gpt-proA] 62, lacyl, tsx-29, glnV44 [AS], galK2 [Oc], LAM-, aroD6, hisG4 [Oc], xylA5, mtl-1, argE3 [Oc], thi-1) and E. coli K12 JE5505 (Lpp⁻; mutations: DE [gpt-proA] 62, lacyl, tsx-29, glnV44 [AS], galK2 [Oc], LAM-, Ipp-254 [del], pps-6, hisG4 [Oc], xylA5, mtl-1, argE3 [Oc], thi-1) were obtained from the E. coli Genetic Stock Center (New Haven, Conn.). Pittard J and Wallace B J, J Bacteriol 91:1494-1500 (1966); Hirota Y et al., Proc Natl Acad Sci USA 74:1417-1420 (1977). Although not isogenic, these two mutants have nearly identical mutation profiles, and differ only in: lpp (the gene encoding murein lipoprotein is deleted in E. coli K12 JE5505), aroD6 (the gene encoding 3-dehydroquinase, a 26 kDa protein, is mutated in the Lpp⁺ strain (Duncan K et al., Biochem J 238:475-483 (1986)), and pps-6 (the gene encoding phosphenolpyruvate synthase, a roughly 84 kDa protein is mutated in the Lpp⁻ strain (Geerse R H et al., Mol Gen Genet 218:348-352 (1989)).

Bacteria were cultured in trypticase soy broth (TSB, Difco, Detroit) from colonies stored on trypticase soy agar (TSA, Difco). Media was supplemented with kanamycin (50 mg/ml) for E. coli K12 CH202pRC2 and ampicillin (100 mg/ml) for E. coli K12 JC7752p417 to maintain the plasmids. Bacteria were cultured at 37° C. with vigorous agitation to the desired growth phase, harvested, and washed by low speed centrifugation in sterile normal saline (5000-8000× g, 8-10 minutes, 4° C.).

### Example 1

Monoclonal Antibodies

Methods. Prior studies indicated that anti-J5 IgG binds three OMPs of MWs 35 kDa, 18 kDa (previously estimated as 37 kDa and 24 kDa respectively: Hellman J et al., J Infect Dis 176:1260-1268 (1997)) and 5-9 kDa, that are present on the bacterial surface and are released into human serum as OMP/LPS complexes. Monoclonal antibodies were prepared against each of the three OMPs bound by IgG in J5 antiserum, and against the O-polysaccharide of E. coli O18 LPS. For production of anti-OMP monoclonal antibodies, BALB/c mice (Charles River Laboratories, Wilmington, Mass.) were immunized with heat-killed, lyophilized E. coli J5 vaccine prepared as described. Siber G R et al., J Infect Dis 152:954-964 (1985). Vaccine was resuspended in sterile normal saline (1 mg/ml). Increasing doses were injected intraperitoneally 3 times per week for three weeks (0.1 mg, 0.2 mg, and 0.3 mg). Booster injections were given monthly for 1-3 months, with the final booster three days prior to harvesting the spleen. Splenocytes were harvested and fused with myeloma cells by standard laboratory protocol. Kohler G et al., Eur J Immunol 6:292-295 (1976); Cold Spring Harbor Laboratory (1988) Antibodies: A Laboratory Manual, Cold Spring Harbor Press, Cold Spring Harbor. Fused cells were cultured in Dulbecco's Modification of Eagle's Medium (DMEM, Mediatech Cellgro) supplemented with glucose (4.5 gm/L), L-glutamine, 20% heat-inactivated fetal calf serum (Mediatech), penicillin (100 units/ml), and streptomycin (100 mg/ml).

The three OMPs are exposed on the surface of bacteria after incubation in human serum. Hellman J et al., J Infect Dis 176:1260-1268 (1997). Accordingly, monoclonal antibodies were initially screened by bacterial ELISA, using heterologous serum-exposed smooth E. coli isolates (E. coli 08:K45:H1, 016:K1:H6, and 025:K5:H1) as the coating antigen, and hybridoma culture supernatants as primary antibody. Hellman J et al., J Inf Dis 176:1260-68 (1997). Bacteria were grown to the desired phase as determined by optical density at 550 nm (A₅₅₀), washed in sterile saline, suspended in serum or saline to an A₅₅₀ of 1.0, and incubated at 37° C. for the specified time (10 minutes-1 hour). The bacteria were washed by centrifugation (5000-8000× g, 8-10 minutes, 4° C.) three times in sterile normal saline and resuspended in an equal volume of carbonate buffer, pH 9.6 (50 mM sodium carbonate; EM Science, Cherry Hill, N.J.). Polyvinyl microtiter plates (Dynatech Laboratories, Chantilly, Va.) were coated with bacteria (10⁸ bacteria/ml) and incubated overnight at 4° C. The microtiter plates were then washed three times (PBS, 1 mg/ml Tween 20, 1 mg/ml bovine serum albumin [BSA], 2 mg/ml MgCi₂), blocked overnight at 4° C. with PBS containing BSA (1 mg/ml), and washed again. Dilutions of either normal rabbit serum (NRS) or rabbit antiserum to E. coli J5 were added and plates were incubated (2 hours, 37° C.). After three additional washings, horseradish peroxidase-conjugated anti-rabbit IgG (Cappel, Durham, N.C.) was added, and the plates were incubated (2 hours, 37° C.) and washed. Peroxidase substrate (1 mg/ml H₂O₂ in ABTS, citric acid, Na₂HPO₄) was added, plates were incubated at room temperature for 30 minutes, and the A₄₀₅ was read (ELISA reader EAR400; SLT Lab Instruments, Hillsborough, N.C.). Titers were determined using a standard curve as previously described. Zollinger W D and Boslego J W, J Immunol Methods 46:129-140 (1981). Standard curves were generated using known concentrations of rabbit IgG (Cappel). All assays were performed in duplicate and mean values determined.

Antibodies that bound to serum-exposed bacteria were then analyzed for binding to the three OMPs by immunoblotting using E. coli O25:K5:H1 bacterial lysates as antigen and supernatants from fusions as primary antibody. Immunoblotting was used to detect binding of antisera and monoclonal antibodies to washed bacteria (10⁶/well) and bacterial antigens that were affinity purified from filtrates of serum exposed bacteria. All samples were prepared in sample buffer (2.5% SDS, 22% glycerol, 0.5% β-mercaptoethanol, and trace bromophenol blue in Tris base). Samples were electrophoresed on 16% SDS-polyacrylamide gels and transferred to nitrocellulose (Bio-Rad Laboratories, Hercules, Calif.) by applying 200 mA of constant current at 4° C. for 1 hour (Hoefer Scientific Instruments, San Francisco). For most experiments, the nitrocellulose was blocked (1 hour at room temperature, or overnight at 4° C.) with 1% powdered skim milk in TTBS (150 mM NaCl, 50 mM Tris, 0.1% Tween-20, pH 7.5), washed for 10-15 minutes with TTBS, incubated with primary antibodies, and washed 3 times. Primary antibodies included IgG in rabbit antisera to heat-killed E. coli J5 and E. coli O18 O-polysaccharide (both diluted 1:500 in TTBS), IgG in mouse antiserum to heat-killed E. coli J5, and murine monoclonal antibodies directed to each of the three OMPs (at a concentration of 1 μg/ml). Blots were then incubated for 30 minutes with biotin-conjugated anti-rabbit or anti-mouse IgG antibody (Vectastain, Vector Laboratories, Burlingame, Calif.) diluted 1:240 in TTBS, washed, and then incubated for 30 minutes in a mixture of avidin and biotinylated horseradish peroxidase complex, as described in the manufacturer's instructions (Vectastain). After a final wash with PBS, peroxidase substrate was added (2 ml of 3 mg/ml 4-chloro-1-naphthol, 8 ml of PBS, 10 microliters of 30% H202). The reaction was stopped after 30 minutes by repeated rinsing with distilled water.

Following initial screening, hybridomas of interest were subcloned by limiting dilution to one cell in every fourth well to derive subclones with strong growth characteristics and high production of the antibodies with the binding characteristics described below. Polyclonal mouse anti-J5 IgG was used as a positive control, and pre-immune serum served as the negative control.

Two methods were used to prepare large amounts of the monoclonal IgGs from the hybridoma cell lines isolated as described above. Monoclonal antibodies directed to each of the three OMPs and to the O-polysaccharide of E. coli O18 LPS (Mab anti-O18 IgG) were produced in ascites of BALB/c mice by mouse hybridoma cell lines. The hybridoma cell line producing Mab anti-O18 IgG was the kind gift of A. Cross. Kim KS et al., J Infect Dis 157:47-53 (1988). Ten days after intraperitoneal instillation of 0.5 ml of Pristane (Sigma, St. Louis, Mo.), 5-10×10⁶ hybridoma cells were collected, washed twice in Hanks' Balanced Salt Solution (Cellgro, Mediatech Inc., Herndon, Va.), and injected intraperitoneally. Ascites was collected by aspiration every 2-3 days three times. Cold Spring Harbor Laboratory (1988) Antibodies: A Laboratory Manual, Cold Spring Harbor Press, Cold Spring Harbor. Monoclonal antibody against the 18 kDa OMP was also produced in an artificial capillary cell culture system (Cellmax, Cellco, Laguna Hills, Calif.). The cartridge (Cellmax 011 module) was inoculated with 2.5×10⁷ viable cells. Culture medium was Dulbecco's Modification of Eagle's Medium (DMEM, Mediatech Cellgro) supplemented with glucose (4.5 gm/L), L-glutamine, 2.5-10% heat-inactivated fetal calf serum (Mediatech), penicillin (100 units/ml), and streptomycin (100 mg/ml). The concentration of IgG produced in the artificial capillary cell culture was 0.3-1.0 mg/ml as determined by ELISA. Anti-OMP antibodies showed no cross-reactivity with LPS or with proteins in human serum by immunoblotting. The Mab anti-O 18 IgG does not cross-react with LPS from other organisms, with the OMPs, or with proteins in human serum by immunoblotting.

IgG was purified from ascites following ammonium sulfate precipitation and from hyperimmune serum. Cold Spring Harbor Laboratory (1988) Antibodies. A Laboratory Manual, Cold Spring Harbor Press, Cold Spring Harbor; Warren HS et al., J Infect Dis 163:1256-1266 (1991); Ge Y et al., J Infect Dis 169:95-104 (1994). Briefly, affinity chromatography was performed by passage over a protein G-Sepharose 4 fast-flow column (Pharmacia, Piscataway, N.J.). Bound IgG was eluted from the column with 0.1 M glycine (pH 2.7) and was immediately neutralized using 1 M Tris buffer (pH 9.0). Purified IgG was dialyzed against PBS (pH 7.2) and stored at −80° C. Protein concentration was determined by ELISA and by absorption at 280 nm. Zollinger WD and Boslego J W, J Immunol Methods 46:129-140 (1981).

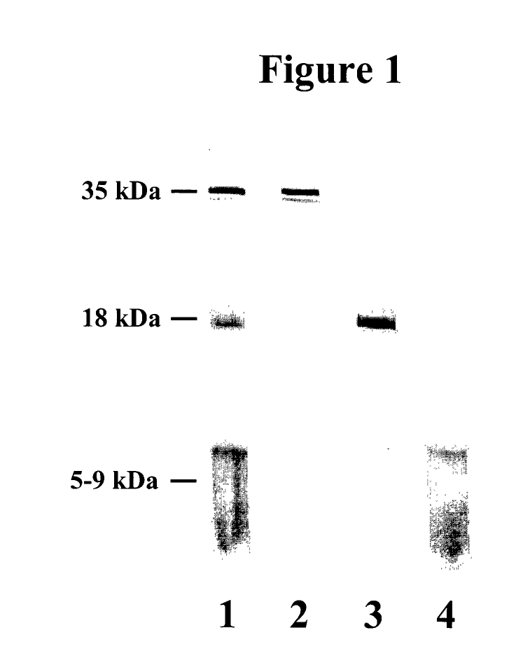

Results. Of 10 splenic fusions, 9 antibodies were identified that bound to the surface of heterologous serum-exposed bacteria by ELISA. Immunoblotting analysis revealed that 7 of the 9 IgGs bound to one of the three OMPs. Three of these anti-OMP monoclonal IgGs (2D3, 6D7, and 1 C7) were selected for increased production, each with specificity for one of the three OMPs. A representative immunoblot of lysates of E. coli O6 bacteria stained with these three monoclonal IgGs and polyclonal anti-J5 IgG is shown in FIG. 1.

Antigen was electrophoresed on a 16% SDS-polyacrylamide gel and transferred to nitrocellulose. Primary antibodies for the immunoblots include polyclonal mouse anti-J5 IgG (lane 1), and three separate monoclonal antibodies, 2D3 (lane 2), 6D7 (lane 3), and 1C7 (lane 4) derived from mice immunized with E. coli J5 vaccine. Estimated molecular weights of the bands (kDa) are indicated at the left of the figure.

### Example 2

Identification of OmpA

We hypothesized that the 35 kDa protein was OmpA based upon the apparent molecular weight and the fact that the electrophoretic mobility of the band was altered by boiling. Hindennach I and Henning U, Eur J Biochem 59:207-213 (1975). Immunoblotting studies were performed to identify this protein.

Recombinant outer membrane protein A (OmpA). The coding region of the 325 amino acid mature OmpA protein, excluding the 21 amino acid signal sequence (GenBank accession #V00307), was generated by PCR amplification of DNA from an extract of E. coli O18:K1:H7. OmpA-specific PCR primers OmpABacl and OmpABac2 contained 5′ extensions for cloning into the transfer plasmid pBACgus-2 cp (Novagen, Madison, Wis.).

OmpABac 1: 5′-GACGACGACAAGGCTCCGAAAGATAACACCTG-3′ (SEQ ID NO: 1)

OmpABac2: 5′-GAGGAGAAGCCCGGTTAAGCCTGCGGCTGAGTTAC-3′ (SEQ ID NO:2)

The transfer plasmid containing the OmpA coding sequence (OmpA/pBACgus-2 cp) was then transfected into the BacVector-2000 Triple Cut Baculovirus DNA in Sf9 cells, according to the manufacturer's instructions (Novagen, Madison, Wis.). Positive recombinants were expanded, and high titer virus was produced, to give multiplicity of infection in the range of 10 to 20 for maximal protein expression in Sf9 cells. The final Baculovirus construct contained the OmpA coding sequence, with an in-frame amino terminal extension (fusion sequences were encoded by the pBACgus-2 cp transfer plasmid) containing an enterokinase recognition sequence, an S-protein binding site and a polyhistidine tail. The 36.5 kDa OmpA fusion protein (calculated molecular weight) was purified from Baculovirus-infected Sf9 cell lysates by polyhistidine affinity chromatography over a Talon cobalt metal affinity resin according to the manufacturer's instructions (Clontech, Palo Alto, Calif.).

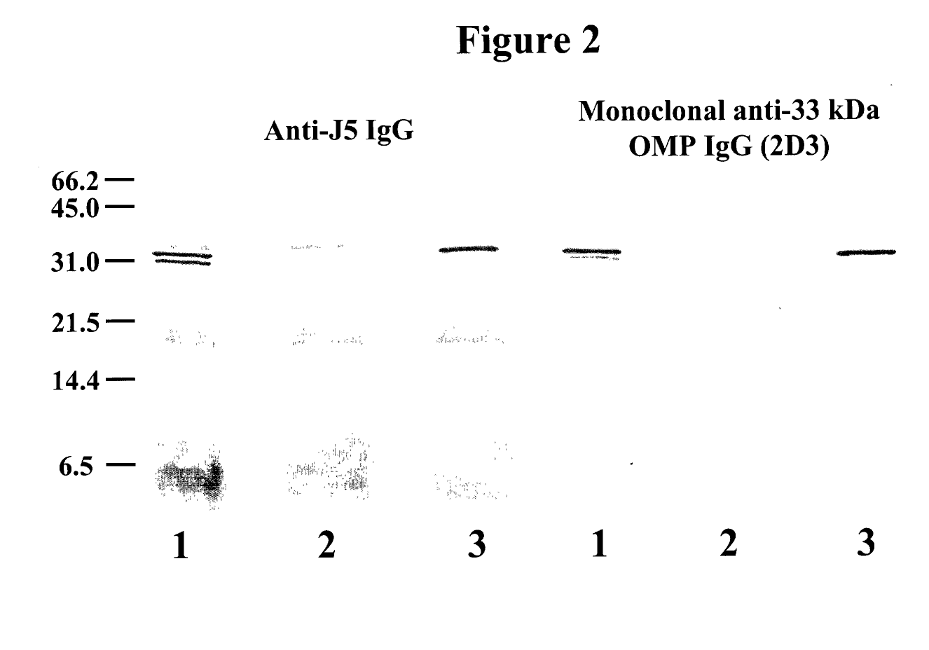

The 35 kDa OMP is OmpA. Isolates of E. coli O18 bacteria in which the OmpA gene was deleted and then replaced back into the strain (Prasadarao N V et al., Infect Immun 64:146-153 (1996)) and recombinant OmpA were electrophoresed on 16% SDS-polyacrylamide gels, transferred to nitrocellulose, and used as antigen in immunoblotting assay performed as described above. Primary staining antibodies included anti-J5 IgG and monoclonal IgG that is directed against the 35 kDa OMP (2D3). Anti-J5 IgG and 2D3 did not react with the 35 kDa band in lysates of bacteria in which the OmpA gene was deleted, but did react with a 35 kDa band in the wild-type strain and the strain in which the gene was reinserted (FIG. 2). Bacterial strains are: wild type OmpA⁺ E. coli O18:K1:H7 (lane 1); E91, an OmpA-deleted mutant of E. coli O18:K1:H7 (lane 2); E69, and an OmpA-restored mutant of E. coli O18:K1:H7 (lane 3). Molecular weight markers (kDa) are as at the left.

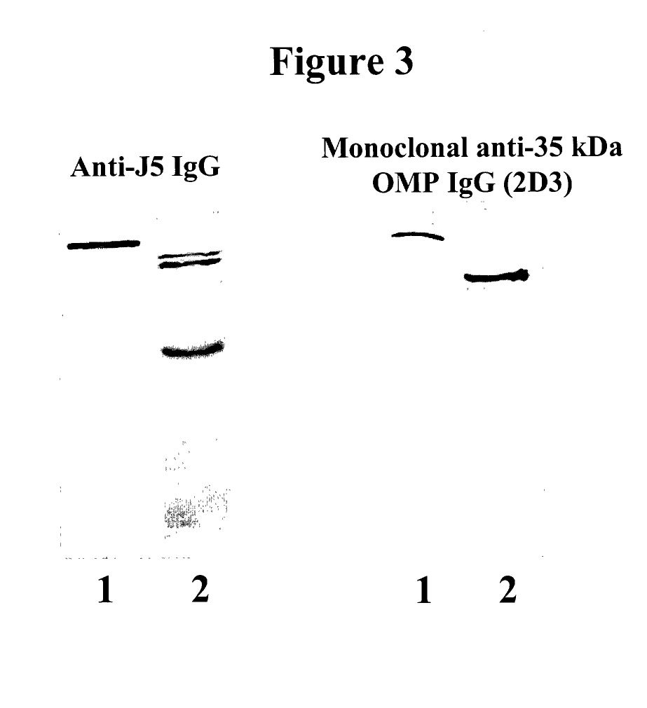

Recombinant OmpA was stained by anti-J5 IgG and 2D3 (FIG. 3). Recombinant OmpA (lane 1 of each panel) and lysates of E. coli O18:K1:H7 bacteria (lane 2 of each panel) were electrophoresed on a 16% SDS-polyacrylamide gel and transferred to nitrocellulose. Primary antibodies included polyclonal mouse anti-J5 IgG (left panel) and the monoclonal antibody 2D3 (right panel). Recombinant OmpA ran at a slightly higher molecular weight, presumably because of the polyhistidine tag that is present on the recombinant protein. These results indicate that the 35 kDa OMP is OmpA and that 2D3 is a monoclonal anti-OmpA IgG.

### Example 3

Identification of PAL

Methods. The final purification procedure for the 18 kDa OMP consisted of: 1) preparation of total bacterial membranes, 2) Triton X-100 extraction of bacterial membranes, 3) affinity chromatography using sepharose beads conjugated with 6D7 (the anti-18 kDa OMP monoclonal antibody), and 4) reverse-phase HPLC separation. The purification steps are described below.

Total bacterial membranes were prepared from mid-late log-phase cultures of E. coli O18K bacteria essentially as described. Hellman J et al., J Infect Dis 176:1260-1268 (1997); Munford RS et al., J Bacteriol 144:630-640 (1980). Unless otherwise indicated, all steps were performed at 4-6° C. 2 L cultures of bacteria were harvested by centrifugation and the resultant pellets were resuspended in a total of 60 ml pre-chilled 10 mM HEPES buffer (pH 7.4) with 25% sucrose (w/v) and 0.2 mM dithiothreitol (DTT, Fisher Biochemicals, Fair Lawn, N.J.). RNase and DNase (Sigma, St. Louis) were each added to a final concentration of 4 μg/ml. Cells were disrupted by sonicating the suspension on ice (microtip, 30-60 second bursts separated by 60-90 seconds, total sonication time 4 minutes). Unbroken bacteria and other debris were removed by centrifugation (10,000× g, 40 minutes), and the supernatant was collected (volume 60 ml). 15 ml of HEPES buffer (pH 7.4) containing EDTA (25 mM), and DTT (0.2 mM) was added to the 60 ml to adjust the concentration of sucrose to 20% (w/v) and the concentration of EDTA to 5 mM. Samples were layered onto a 60% (w/v) sucrose cushion (7.5 ml sample per 4.5 ml cushion) and ultracentrifuged (100,000× g, 3 hours, 6° C.). Bacterial membranes present in the hazy white/yellow band at the interface were collected by puncturing the side of the tube with a 20 gauge needle and aspirating gently with a 1 ml syringe (approximately 0.5 ml/tube, final volume 5 ml). Total membranes were dialyzed against Tris-HCI (20 mM, pH 8) overnight (2 L) and then against fresh buffer for 48 hours (4 L). The final volume of dialyzed material was approximately 15 ml/2 L of the starting bacterial culture.

Sixty ml of dialyzed total membranes representing 8 liters of the starting bacterial culture were concentrated to 36 ml using a nitrogen pressurized system and a Diaflo ultrafiltration membrane, YM30 filter (Millipore Company, Danvers, Mass.) according to the manufacturer's instructions, and extracted with Triton X-100. Twelve ml of a stock solution of 10% Triton X-100 in Tris-HCI (20 mM, pH 8.4), containing the protease inhibitor 4-(2-aminoethyl)-benzenesulfonyl fluoride (Sigma) and EDTA were added to the membranes (final concentrations: 2.5% Triton X-100, 0.5 mM 4-(2-aminoethyl)-benzenesulfonyl fluoride, 5 mM EDTA). The sample was incubated at room temperature for 30 minutes, and then ultracentrifuged (TH641 swinging bucket rotor, 100,000× g, 2 hours, 6° C.). The resultant supernatant (48 ml) was saved.

The detergent-extracted membrane supernatant was circulated overnight at 9-10 ml/hour through a 5.5 ml column of mouse monoclonal IgG (6D7) directed against the 18 kDa OMP covalently conjugated to CNBr-activated Sepharose 4B beads (4° C.). Unbound material was washed from the column with 36 ml of 2.5% Triton X-100 in 200 mM NaPhos, 0.5 M NaCl, pH 6.8. Bound antigen was eluted with increasing concentrations of SDS (0.125, 0.25, 0.5, and 1%, in 200 mM phos, 0.5 M NaCl, pH 6.8). Three milliliters of each concentration of SDS was applied to the column followed by 9 ml of wash buffer. The protein was detected in 0.5% and 1% SDS-eluted samples. Material eluted with 0.5% and 1% SDS were combined and concentrated to 4 ml by centrifugation in a Centricon Plus-20 centrifugal filter device (10 kDa cutoff, Biomax-8 series, Millipore Corporation).

Three milliliters of the concentrated affinity-purified sample was applied to an analytical C4 reverse-phase HPLC column (Vydac, Hesperia, Calif.) and eluted using a linear gradient of 5-95% acetonitrile/0.1% trifluoroacetic acid/H₂O at a flow rate of 1 ml/min. Fractions were collected at one minute intervals into 20 microliters of 2-fold concentrated SDS-PAGE sample buffer (5% SDS, 44% glycerol in Tris base) and lyophilized. Lyophilized samples were resuspended in 40 microliters of water with β-mercaptoethanol (0.5%) and trace bromophenol blue and heated (100° C., 5-10 minutes). Fractions were electrophoresed and analyzed for the 18 kDa OMP by immunoblotting using anti-J5 IgG or 6D7 (the monoclonal anti-18 kDa OMP IgG) as the primary antibody.

The peak fraction from the C4 HPLC separation was electrophoresed on a 16% SDS-polyacrylamide gel and stained with Coomassie brilliant blue. The faintly staining 18 kDa band was then cut from the gel, washed twice (50% acetonitrile, 0.5 ml, 3 minutes) and frozen. Sequence analysis of two peptides of a trypsin digestion of the protein in the gel was performed at the Harvard Microchemistry Facility by tandem mass spectrometry (MS/MS) on a Finnigan LCQ quadrupole ion trap mass spectrometer.

The 18 kDa OMP is PAL. Two peptide sequences (Sequence 1 and Sequence 2, 10 and 14 amino acids, respectively) were obtained that each mapped with 100% homology to PAL.

| | | |
|---|---|---|
| Sequence 1: | VTVEGHADER | (SEQ ID NO:3) |
| Sequence 2: | [G][V]SADQ*I*VSYGK* | (SEQ ID NO:4) |

Brackets ([ ]) indicate that the amino acid has been identified with reasonable confidence. Stars (*) indicate that the amino acid is isobaric and cannot by unambiguously differentiated by mass spectrometric sequencing. All other amino acids were assigned with the highest confidence.

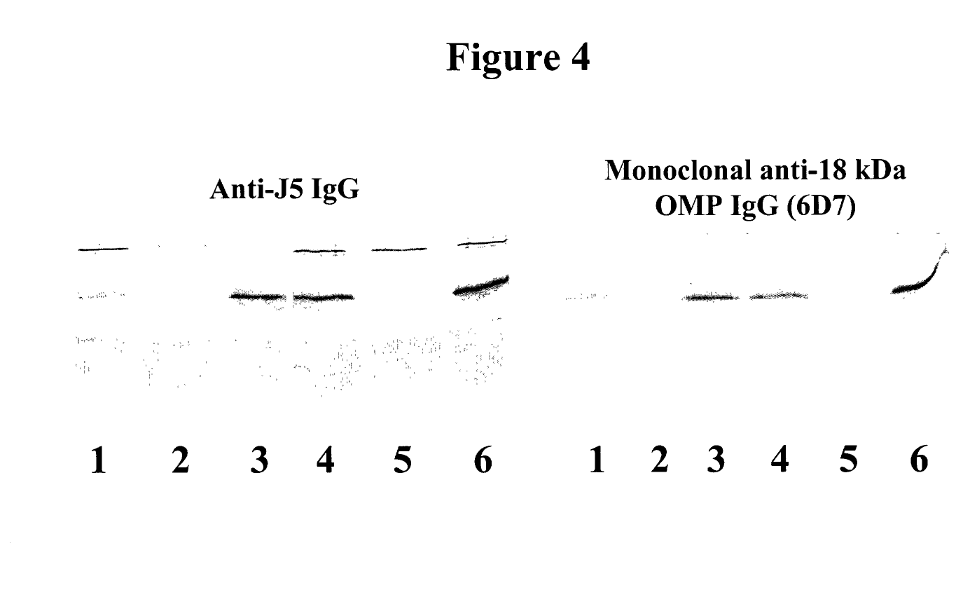

The identity of PAL was confirmed by immunoblotting studies. Referring to FIG. 4, lysates of E. coli K12 bacteria in which the PAL gene (excC) was deleted, or was deleted and then replaced, were immunoblotted using with anti-J5 IgG (left panel) or monoclonal anti-18 kDa OMP IgG 6D7 (right panel) as primary antibody. Bacterial strains in both panels of FIG. 4 are: E. coli K12 p400 containing PAL (lane 1); CH202, a PAL-deficient mutant of E. coli K12 p400 (lane 2); CH202 prC2, a PAL-restored mutant of CH202 (lane 3); E. coli K12 1292 containing PAL (lane 4); JC7752, a PAL-deficient mutant of 1292 (lane 5); and JC7752 p417, a PAL-restored mutant of JC7752 (lane 6). Anti-J5 IgG and 6D7 did not react with the 18 kDa band in lysates of PAL-deficient bacteria, but did react with an 18 kDa band in the wild-type strain and the strain with the gene reinserted (FIG. 4). These results indicate that the 18 kDa OMP is PAL, and that 6D7 is a monoclonal anti-PAL antibody.

### Example 4

Identification of MLP

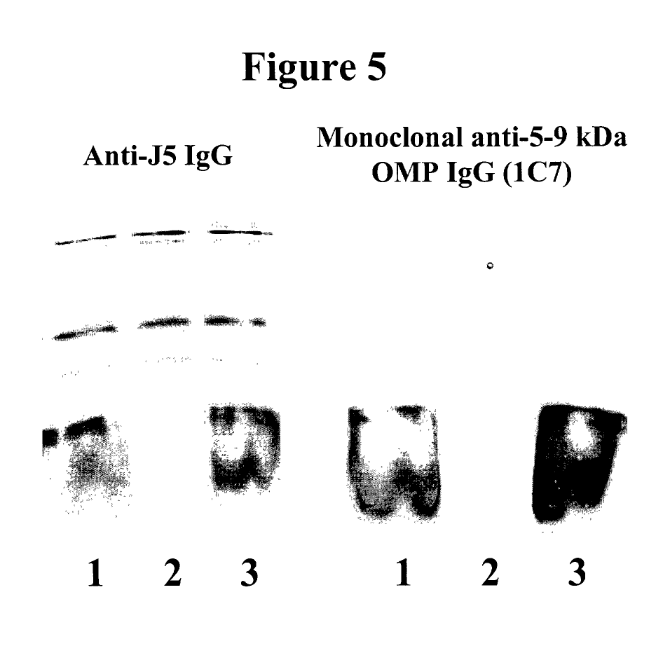

The 5-9 kDa OMP is murein lipoprotein (MLP). We hypothesized that the 5-9 kDa OMP was MLP based on its low molecular weight and size heterogeneity. Hantke K and Braun V, Eur J Biochem 34:284-296 (1973). Accordingly, an isolate of E. coli K12 in which the murein lipoprotein (lpp) gene was deleted, a very closely related mutant strain containing MLP, and the standard laboratory strain, E. coli O18 (also containing MLP), were used as antigens on identical immunoblots. Referring to FIG. 5, the immunoblot in the left panel was developed with anti-J5 IgG, and the immunoblot in the right panel was developed with the monoclonal IgG that binds to the 5-9 kDa OMP (1C7). Various lanes in the two immunoblots shown in FIG. 5 correspond to: E. coli O18K⁺, containing MLP (lane 1); MLP-deficient E. coli K12 JE5505 (lane 2); and closely related E. coli K12AT1360, containing MLP (lane 3). Anti-J5 IgG and 1 C7 IgG did not react with the 5-9 kDa band in bacterial lysates of the MLP-deficient strain (lane 2). These results demonstrate that the lower molecular weight cross-reactive OMP is MLP and that monoclonal antibody 1 C7 reacts with MLP.

As mentioned above, the mutation profiles of the Lpp⁺ and Lpp⁻ E. coli K12 isolates are nearly identical, differing in the deletion of lpp (the gene encoding MLP) and mutations in aroD6 (the gene encoding a 26 kDa protein) in the Lpp+isolate, and pps-6 (the gene encoding an 84 kDa protein) in the Lpp isolate. Pittard J and Wallace B J, J Bacteriol 91:1494-1500 (1966); Hirota Y et al., Proc Natl Acad Sci USA 74:1417-1420 (1977). The unmutated gene products of aroD and pps-6 have molecular weights that are considerably higher than that of MLP (26 and 84 kDa respectively, versus 5-9 kDa for MLP) and are not described to exhibit the same heterogeneity of molecular weight that is exhibited by MLP. Duncan K et al., Biochem J 238:475-483 (1986); Geerse R H et al., Mol Gen Genet 218:348-352 (1989). Thus it is doubtful that the difference in the pattern of staining is due to the mutations other than lpp.

### Example 5

Identification of OMPs Released by Bacteria Incubated in Human Serum

Previous studies have demonstrated that E. coli and Salmonella bacteria incubated in human serum release complexes of OMPs and LPS that can be affinity-purified using O-chain specific anti-LPS IgG. Hellman J et al., J Infect Dis 176:1260-1268 (1997); Freudenberg M A et al., Microbial Pathogenesis 10:93-104 (1992). To test the hypothesis that OmpA, PAL, and MLP are present in OMP/LPS complexes released by bacteria into human serum, polyclonal anti-O18 IgG was used to affinity-purify LPS from sterile filtrates of human serum incubated with E. coli O18K⁺ bacteria as described.

Methods. The following IgGs were covalently conjugated to magnetic beads (BioMag Amine Terminated 8-4100, PerSeptive Diagnostics, Cambridge, Mass.) according to the manufacturer's instructions and as previously described (Hellman J et al., J Infect Dis 176:1260-1268 (1997)): murine monoclonal IgG directed against the O-polysaccharide of E. coli O18 LPS and an unrelated murine IgGI (ATCC, Rockville, Md.), IgG from rabbit antisera to the E. coli O18 O-polysaccharide vaccine and to heat killed E. coli J5, and IgG from normal rabbit serum (normal rabbit IgG). Briefly, magnetic beads were activated by incubation in 5% glutaraldehyde, washed and incubated with dialyzed IgG at 5 mg IgG/ml. The percentage of IgG covalently coupled to the beads was 85-95%. Hellman J et al., J Infect Dis 176:1260-1268 (1997).

E. coli O18K⁺ bacteria were grown to mid-log phase, harvested and washed. The resultant bacterial pellet was resuspended in an equal volume of normal human serum (10⁸ bacteria/ml) with ampicillin (200 μg/ml) and incubated for 2 hours at 37° C. on a rotating drum. The serum was filtered through a 0.45 micron filter to remove intact bacteria. The serum filtrate was then incubated with antibody-conjugated magnetic beads. Antibodies used for these affinity-purification studies included: polyclonal anti-O18 IgG, IgG from J5 antiserum, and IgG from normal rabbit serum. Two hundred microliters of each sample was incubated with IgG-conjugated beads that had previously been washed and resuspended in 800 microliters of PBS (final concentration of IgG 100 μg/ml). Reaction mixtures were incubated for 16-20 hours at 4° C., with end-over-end mixing. The antibody-conjugated beads with attached antigens were then separated from the 1:4 diluted serum by placing the tubes in a strong magnetic field, and the beads were washed three times with PBS. Antigen was eluted by heating the beads (5 minutes, 100° C.) in 100 microliters SDS-PAGE sample buffer (2.5% sodium dodecylsulfate, 22% glycerol, in Tris base). Supernatants were carefully separated from the beads, and β-mercaptoethanol (0.5%) and trace bromophenol blue were added. Twenty microliters of each sample were then electrophoresed on lanes of 16% gels and transferred to nitrocellulose. Blots were stained with mouse anti-J5 IgG, Mab anti-O 18 IgG, and mouse monoclonal antibodies directed against each of the OMPs (2D3, 1C7, and 6D7). Blots were developed as described above using biotinylated horse anti-mouse IgG as secondary antibody.

Captured antigens were immunoblotted with the murine monoclonal IgGs against OmpA (2D3), MLP (1C7), and PAL (6D7), Mab anti-O18, and murine polyclonal anti-J5 IgG.

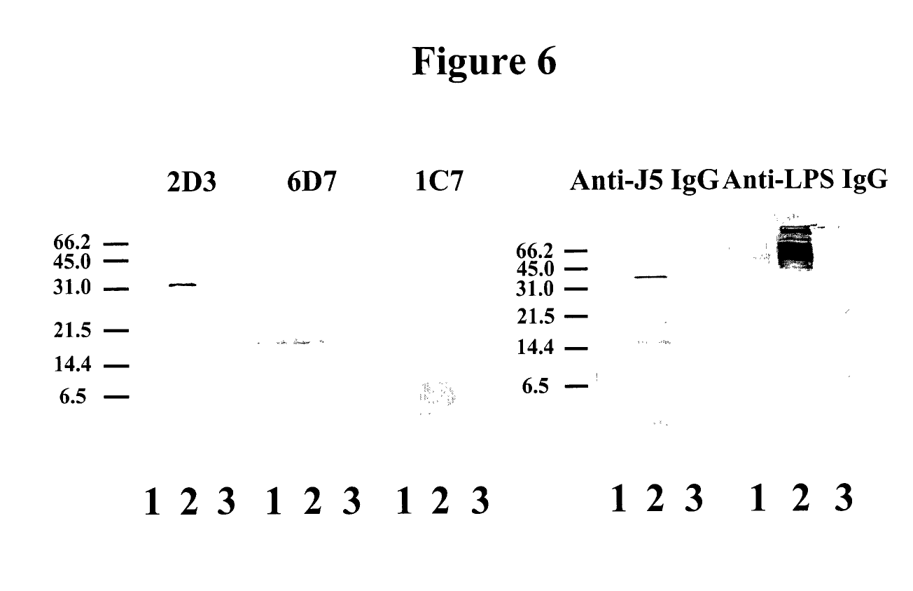

Results. OmpA, PAL, and MLP were all detected in samples that were affinity-purified using polyclonal anti-O 18 IgG, indicating that bacteria release complexes containing these OMPs and LPS. Referring to FIG. 6, eluted samples stained with murine monoclonal IgGs directed against OmpA (2D3), PAL (6D7), and MLP (1C7) are shown in the three left panels, and samples stained with polyclonal mouse anti-J5 IgG and a murine monoclonal IgG directed to the O-polysaccharide chain of E. coli O18 LPS are shown in the two right panels. Samples in the various lanes correspond to those affinity-purified with: rabbit anti-J5 IgG (lane 1), rabbit O-chain specific anti-LPS IgG (lane 2), and normal rabbit IgG (lane 3). Molecular weight markers are as indicated to the left of the two sets of panels. PAL, but not OmpA or MLP, was also detected in samples that were affinity-purified using anti-J5 IgG (FIG. 6). The OMPs were not detected in immunoblots of samples that were affinity-purified using IgG from normal rabbit serum. The OMPs were also not detected in immunoblots of samples prepared from sterile filtrates of bacteria incubated with ampicillin without human serum.

### Example 6

Model of Gram-Negative Sepsis in Burned Rats

Release of OMPs was studied in an infected burn model in rats that was adapted from a murine sepsis model. Stevens E J et al., A quantitative model of invasive Pseudomonas infection in burn injury, J Burn Care Rehabil 15:232-235 (1994).

Methods. Male Sprague-Dawley rats weighing 225-250 gm were anesthetized with ether (Sigma) and subjected to a 15% total body surface area full-thickness burn by application of heated brass bars (100° C., 15 seconds). Rats were then inoculated by subcutaneous injection of E. coli O18K⁺ (10-100 CFU) into the burned area. At 72 hours, all rats were bacteremic and were given an intravenous dose of ceftazidime (80 mg/kg) via the tail vein. Blood was collected into 5 mM EDTA (to prevent coagulation) by cardiac puncture 3 hours later and diluted four-fold with PBS. Plasma was prepared by centrifugation (200× g, 5 minutes, 4° C.) and then filtered (0.45 micron) to remove intact bacteria.

Filtered plasmas from septic rats were incubated with magnetic beads covalently conjugated with polyclonal rabbit anti-J5 IgG, antigen-nonspecific control IgG, and anti-O-chain specific IgG. Antibody-conjugated beads were washed and resuspended in 500 microliters of filtered rat plasma (final concentration of IgG 100 μg/ml), incubated overnight, and washed with PBS as described above. Antigen was eluted by heating beads in 50 microliters SDS-PAGE sample buffer, and samples were further processed as described above. Twenty microliters of each sample containing eluted antigen was electrophoresed on 16% SDS-polyacrylamide gels and transferred to nitrocellulose. Captured bacterial antigens were assessed for the three OMPs by immunoblotting using a mixture of murine monoclonal antibodies (2D3, 6D7, and 1 C7) directed against each of the three OMPs as the primary antibody and developing by the more sensitive chemiluminescence method. Following transfer, nitrocellulose was blocked with 5% powdered skim milk in TTBS, washed, and then incubated with primary and secondary antibodies, and with avidin-biotin-peroxidase as described above. Blots were then rinsed three times with TTBS and developed using equal volumes (1-2 ml each) of enhanced luminol and oxidizing reagents (Renaissance Chemiluminescence Reagents, NEN Lifesciences Products, Boston, Mass.). Film (Reflection Autoradiography, NEN Lifesciences Products) was exposed for 30 seconds to 1 minute.

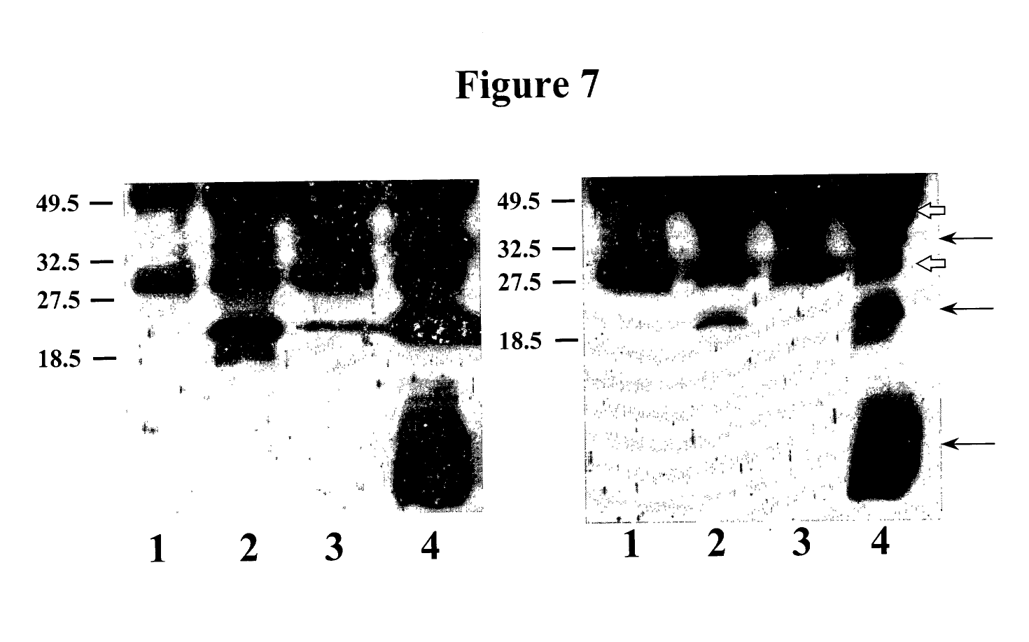

Results. Representative data obtained from two rats are shown in the blots presented in FIG. 7. Three bands were present in samples affinity-purified by anti-O chain specific IgG in 3 of 9 rats, and at least 1-2 bands were present in 6 of 9 rats. Lanes in FIG. 7 correspond to: plasma collected from bacteremic rats immediately prior to (lane 1) and 3 hours after (lanes 2, 3, 4) intravenous administration of ceftazidime. Filtered plasmas were affinity purified with polyclonal rabbit anti-J5 IgG (lanes 1 and 2), normal rabbit IgG (lane 3), and polyclonal rabbit IgG directed against the O-polysaccharide side chain of E. coli O18 LPS (lane 4). The black arrows to the right of the blots point to the 5-9 kDa, 18 kDa, and 35 kDa OMPs. The blots were developed by the more sensitive chemiluminescence technique which amplifies cross-reactive IgG bands (denoted by white arrows to right of the figure). The 18 kDa OMP was present in samples affinity purified using anti-J5 IgG (lanes 1 and 2) in 7 of 9 rats. In 2 of 9 rats there was also some capture of the 5-9 kDa and 35 kDa OMP by anti-J5 IgG.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A vaccine comprising an effective amount of an isolated outer membrane protein selected from the group consisting of OmpA, PAL, MLP, and any immunogenic portion thereof, in a pharmaceutically suitable carrier.

2. The vaccine of claim 1 further comprising an adjuvant.

3. The vaccine of claim 2 wherein the adjuvant is selected from the group consisting of Al(OH)₃, AlPO₄, QS21, CpG, and any combination of these.

4. The vaccine of claim 1 wherein the isolated outer membrane protein is OmpA.

5. The vaccine of claim 1 wherein the isolated outer membrane protein is PAL.

6. The vaccine of claim 1 wherein the isolated outer membrane protein is MLP.

7. An adjuvant comprising an effective amount of an isolated outer membrane protein selected from the group consisting of OmpA, PAL, MLP, and any combination thereof, in a pharmaceutically suitable carrier.

8. A pharmaceutical composition for treating a subject infected with Gram-negative bacteria, comprising an effective amount of an isolated polypeptide that binds specifically to at least a portion of an outer membrane protein selected from the group consisting of OmpA, PAL, and MLP, in a pharmaceutically suitable carrier.

9. The composition of claim 8 wherein the polypeptide is a monoclonal antibody.

10. The composition of claim 8 wherein the polypeptide comprises a fragment of a monoclonal antibody.

11. The composition of claim 8 wherein the polypeptide is a polyclonal antibody.

12. The composition of claim 8 wherein the polypeptide is a member of a combinatorial library of synthetic polypeptides.

13. The composition of claim 9 wherein the monoclonal antibody is a human monoclonal antibody.

14. The composition of claim 9 wherein the monoclonal antibody is a humanized monoclonal antibody.

15. The composition of claim 10 wherein the monoclonal antibody is a human monoclonal antibody.

16. The composition of claim 10 wherein the monoclonal antibody is a humanized monoclonal antibody.

17. An immortal cell line which secretes a polypeptide that binds specifically to an outer membrane protein selected from the group consisting of OmpA, PAL, MLP, and any immunogenic portion thereof.

18. The immortal cell line of claim 17 wherein the polypeptide is a monoclonal antibody.

19. The immortal cell line of claim 17 wherein the polypeptide comprises a fragment of a monoclonal antibody.

20. The immortal cell line of claim 17 wherein the outer membrane protein is OmpA.

21. The immortal cell line of claim 17 wherein the outer membrane protein is PAL.

22. The immortal cell line of claim 17 wherein the outer membrane protein is MLP.

23. The immortal cell line of claim 18 wherein the monoclonal antibody is a human antibody.

24. The immortal cell line of claim 18 wherein the monoclonal antibody is a humanized antibody.

25. A method of immunizing a subject against infection due to Gram-negative bacteria comprising:
administering to a subject an isolated outer membrane protein antigen selected from the group consisting of OmpA, PAL, MLP, and any immunogenic portion thereof, in a pharmaceutically suitable carrier, in an amount effective for inducing protection against infection due to Gram-negative bacteria.

26. The method of claim 25 wherein the antigen is OmpA.

27. The method of claim 25 wherein the antigen is PAL.

28. The method of claim 25 wherein the antigen is MLP.

29. The method of claim 25 further comprising the administration of an adjuvant.

30. The method of claim 29 wherein the adjuvant is selected from the group consisting of Al(OH)₃, AlPO₄, QS21, CpG, and any combination thereof.

31. The method of claim 25 wherein the antigen is administered subcutaneously.

32. The method of claim 25 wherein the antigen is administered intradermally.

33. The method of claim 25 wherein the antigen is administered mucosally.

34. The method of claim 25 wherein the antigen is administered intramuscularly.

35. A method of treating a subject who has an infection with Gram-negative bacteria comprising:
administering to a subject who has an infection with Gram-negative bacteria an isolated polypeptide that binds specifically to at least a portion of an outer membrane protein selected from the group consisting of OmpA, PAL, and MLP, in an amount effective to treat the infection.

36. The method of claim 35 wherein the amount is effective to inhibit Gram-negative sepsis.

37. The method of claim 35 wherein the amount is effective to inhibit growth of the Gram-negative bacteria in vivo.

38. The method of claim 35 wherein the polypeptide is a monoclonal antibody.

39. The method of claim 35 wherein the polypeptide comprises a fragment of a monoclonal antibody.

40. The method of claim 35 wherein the polypeptide is a member of a combinatorial library of synthetic polypeptides.

41. The method of claim 35 wherein the administered amount of polypeptide is effective to enhance clearance of Gram-negative bacteria from blood of the subject.

42. The method of claim 35 wherein the administered amount of polypeptide is effective to enhance clearance of insoluble fragments of Gram-negative bacteria from blood of the subject.

43. The method of claim 35 wherein the administered amount of polypeptide is effective to neutralize Gram-negative bacteria in blood of the subject.

44. The method of claim 35 wherein the administered amount of polypeptide is effective to neutralize insoluble fragments of Gram-negative bacteria in blood of the subject.

45. The method of claim 35 wherein the administered amount of polypeptide is effective to opsonize Gram-negative bacteria in blood of the subject.

46. The method of claim 35 wherein the administered amount of polypeptide is effective to opsonize insoluble fragments of Gram-negative bacteria in blood of the subject.

47. The method of claim 35, further comprising administration of an effective amount of an immune system stimulant.

48. The method of claim 47 wherein the immune system stimulant is a cytokine.

49. The method of claim 47 wherein the immune system stimulant is an adjuvant.

50. A method of treating a subject who has Gram-negative sepsis comprising:
administering to a subject in need of such treatment a composition comprising an isolated polypeptide that binds specifically to at least a portion of an outer membrane protein selected from the group consisting of OmpA, PAL, and MLP, in an amount effective to inhibit sepsis-related release of at least one soluble factor into blood or tissue of the subject.

51. The method of claim 50 wherein the at least one soluble factor is released by Gram-negative bacteria upon exposure of the Gram-negative bacteria to serum.

52. The method of claim 51 wherein the at least one soluble factor is LPS.

53. The method of claim 51 wherein the at least one soluble factor is OmpA.

54. The method of claim 51 wherein the at least one soluble factor is PAL.

55. The method of claim 51 wherein the at least one soluble factor is MLP.

56. The method of claim 50 wherein the at least one soluble factor is a cytokine.

57. The method of claim 50 wherein the at least one soluble factor is a factor selected from the group consisting of TNF-α, MIF, chemokines, and nitric oxide.

58. A method of treating a subject who has Gram-negative sepsis comprising:
administering to a subject in need of such treatment a composition comprising an isolated polypeptide that binds specifically to at least a portion of an outer membrane protein selected from the group consisting of OmpA, PAL, and MLP, in an amount effective to enhance clearance of at least one sepsis-related soluble factor released by Gram-negative bacteria into blood of the subject.

59. The method of claim 58 wherein the soluble factor is LPS.

60. The method of claim 58 wherein the soluble factor is OmpA.

61. The method of claim 58 wherein the soluble factor is PAL.

62. The method of claim 58 wherein the soluble factor is MLP.
